(19) United States
(12) Patent Application Publication
(10) Pub. No.: US 2024/0242833 A1
(43) Pub. Date: Jul. 18, 2024

(54) METHODS AND SYSTEMS FOR THE CLASSIFICATION OF SUBJECTS INTO GLUCOSE HOMEOSTASIS PHENOTYPES

(71) Applicant: KLICK INC., Toronto (CA)

(72) Inventors: Yan FOSSAT, Toronto (CA); Jaycee Morgan KAUFMAN, Toronto (CA); Eric Sin Hang NG, Toronto (CA); Lennaert VAN VEEN, Toronto (CA)

(21) Appl. No.: 18/289,484

(22) PCT Filed: May 5, 2022
(86) PCT No.: PCT/CA2022/050706

§ 371 Date: Nov. 3, 2023

Related U.S. Application Data
(60) Provisional application No. 63/184,560, filed on May 5, 2021.

(51) Int. Cl. G16H 50/20; G16H 50/30

(52) U.S. Cl. CPC G16H 50/20; G16H 50/30

(57) ABSTRACT

Described are methods and systems for classifying a subject into a glucose homeostasis phenotype such as a prediabetic subphenotype based on modelling glycemic control and glucose homeostasis in the subject. Also described is a model of glucose homeostasis based on proportional and integral terms in a control system. A representative curve is generated based on glucose time series data and fit to the model in order to determine coefficients for each subject. The coefficients provide a digital biomarker of glycemic control for the subject and may be used to classify subjects into different glucose homeostasis phenotypes.

### RELATED APPLICATIONS

The present application claims the benefit of priority of U.S. provisional application No. 63/184,560 filed May 5, 2021, the entire content of which is hereby incorporated by reference.

### FIELD

The described embodiments relate to glucose homeostasis and more specifically to methods and systems for classifying a subject into a glucose homeostasis phenotype.

### BACKGROUND

Traditionally, the field of medicine has defined the traits that contribute to “health” as single, discrete values, or set ranges, often taken at a single time point (Brüssow, 2013). This is especially true for many physiological functions, such as glycemia, temperature, body mass index, bone density, cholesterol, blood pressure, etc. These values are measured and assessed using simple scoring gradients where any patient whose value falls into a particular range may be defined as “healthy”, and all others defined as “unhealthy”.

Although using simple heuristics to measure and assess health may be efficient and unambiguous, this approach does not explain the fundamental control mechanisms and physiological systems that lead to these healthy values. Single values only measure the “what” of health and miss the “how”. For example, a blood pressure of 120/80 mm Hg may indicate a “healthy” value, but it is only taken at one static time point in the patient's day. This value gives no indication of how effective the body is at controlling blood pressure when handling physical or mental stress. In other words, the discrete, single time point values of physiological biometrics are merely manifestations of a deeper, more complex health control system.

There is a need therefore to identify, measure and assess the body's ability to maintain homeostasis (i.e., the maintenance of specific variables within an optimal range, regardless of external stimuli) (Kotas & Medzhitov, 2015). For example, many of today's most prevalent chronic illnesses, such as hypertension, diabetes, obesity, and depression, can be considered failures of the body's ability to maintain homeostasis or keep physiological signals within a normal working range.

One approach to define and monitor health involves understanding glucose level variations and normal glycemic control; a dysfunction of this model results in type 2 diabetes (T2D). It is estimated that more than 30 million Americans have T2D, while another 84 million have prediabetes (Centers for Disease Control and Prevention National Diabetes Statistics Report, 2017). Diabetes is also associated with $327 billion of direct and indirect medical costs every year (American Diabetes Association Statistics about Diabetes, 2018). Thus, an evaluation method to understand a patient's glycemic homeostatic function is desired to reduce the economic and social burden of diabetes.

The standard methodology of measuring glycemic dysfunction includes, HbA1C measurements, fasting blood glucose test, and the oral glucose tolerance test (Handelsman, 2015). All three tests use simple heuristics to distinguish healthy patients from those with prediabetes or diabetes. A better evaluation method to understand the nuanced structure of the glycemic system may be obtained by modelling its dynamic function. Although models of normal glycemic control currently exist, they tend to be fairly complicated. These models use a large number of variables and parameters, and describe a multitude of biophysical processes, rather than the resulting control strategy itself. For instance, the model recently proposed by Masroor et al. (2019) comprises 5 dynamical equations and over 25 parameters. The use of such models is limited by the curse of dimensionality, i.e. the catastrophic growth of the number combinations of parameter values to explore when attempting to reproduce measured data.

Monitoring and treating patients at risk of diabetes such as those with prediabetes presents a significant burden on healthcare systems. Furthermore, standard methodologies for measuring glycemic dysfunction do not necessarily provide insights into disease progression or provide guidance with respect to what interventions may be most suitable for a particular case. There remains a need for systems and methods for evaluating glycemic control and glucose homeostasis and classifying subjects at risk of diabetes or with prediabetes.

### SUMMARY

In one aspect, methods and systems are provided for classifying a subject into a glucose homeostasis phenotype. It has surprisingly been determined that glucose homeostasis metrics generated by a model that describes glucose homeostasis as a control system are able to classify subjects into different phenotypes associated with different underlying physiological states. Notably, the systems and methods described herein are not based on traditional risk factors associated with diabetes such as body mass index, activity levels or family history, but rather an empirical analysis of the control of blood glucose levels over time.

As described herein, embodiments include monitoring blood glucose levels, such as by using a continuous glucose monitoring (CGM) device, to generate a representative curve for a subject and fitting the data to a control model, such as a proportional-integral controller equation, and a differential equation describing glucose response. The coefficients of the control model may include a proportional coefficient A₁ for response of a controller u(t) to an error e(t), an integral coefficient A₂ for the response of the controller u(t) to past values of error e(t), an inverse memory time scale/for decay of an integral term, a steady depletion coefficient A₃ for the basic metabolic rate, and a feedback coefficient A₄ for the approximate mass action rate.

As demonstrated in the Examples, homeostasis function is impaired in both prediabetic and diabetic states. Remarkably, this homeostasis impairment has been shown to manifest in several distinct clusters (phenotypes) in prediabetics. Accordingly, the embodiments described herein provide a new dimension for classifying and characterizing subjects with glycemic dysfunction such as prediabetes into distinct phenotypes and not merely for identifying prediabetic subjects. Classification of subjects into different phenotypes associated with different underlying physiological control of glucose may then be used to inform the clinical management and treatment of subjects with glycemic dysfunction such as prediabetes.

For example, the glucose homeostasis metric A₁ has been demonstrated to be useful for classifying subjects into different instantaneous glucose response phenotypes. In some embodiments, male subjects with a value of A₁ less than about 0.25 are classified as having a weak instantaneous glucose response phenotype while female subjects with a value A₁ less than about 0.30 are classified as having a weak instantaneous glucose response phenotype.

Alternatively, or in addition, subjects may be classified into phenotypes using the glucose homeostasis metric A₂/λ. In some embodiments, subjects with a value of A₂/λ less than 0.6 are classified as having a weak inertial glucose response phenotype.

In one embodiment, the methods and systems described herein further comprise selecting a subject for treatment and/or for monitoring based on the classification of the subject into a particular phenotype. In one embodiment, the methods and systems described herein further comprise treating and/or monitoring a subject classified as having a glucose homeostasis phenotype, optionally as having a particular prediabetic subphenotype.

In one aspect, the methods and systems described herein for classifying a subject into a glucose homeostasis phenotype are based on systems and methods for evaluating glucose homeostasis and determining a glucose homeostasis metric.

Accordingly, also provided are systems and methods for evaluating glucose homeostasis. As described herein, a representative curve for a subject is generated using a plurality of curve intervals comprising glucose levels from the subject over time. In one embodiment, the representative curve comprises an interval representative of increasing glucose levels in the subject, a peak and an interval of decreasing glucose levels in the subject.

The representative curve may be analyzed in order to extract information useful for evaluating glycemic control and glucose homeostasis in the subject and optionally classify the subject into a glucose homeostasis phenotype. For example, in one embodiment the representative curve may be compared to one or more controls representative of subjects without glycemic dysfunction. In one embodiment, the representative curve may be compared to one or more controls representative of subjects with a glycemic dysfunction, such as type II diabetes.

Also provided is a model that describes glucose homeostasis as a control system. The control model may comprise a proportional-integral controller equation, and a differential equation describing glucose response. In at least one embodiment of the system, a model may be used to determine the rate of change of blood sugar deviation from a set point, and may incorporate three parameters: A₃ which represents a steady depletion modeling the basic metabolic rate, F(t) which models food intake and circadian rhythm, and A₄ which models feedback from a control system and is based on mass action kinetics. In at least one embodiment, the control system is modelled using a controller function that may include a proportional term with amplitude A₁ which responds proportionally to the deviation from a set point blood sugar level, and an integral term with amplitude A₂ based on the history deviations from the set point blood sugar level. The coefficients of the control model may include a proportional coefficient A₁ for response of a controller u(t) to an error e(t), an integral coefficient A₂ for the response of the controller u(t) to past values of error e(t), an inverse memory time scale λ for decay of an integral term, a steady depletion coefficient A₃ for the basic metabolic rate, and a feedback coefficient A₄ for the approximate mass action rate. The control model may further comprise F(t) which models food intake and circadian rhythm.

In one embodiment, a model of glucose homeostasis for a subject is generated based on the representative curve of the subject and the model of glucose homeostasis as a control system. The representative curve may be determined based on a plurality of glucose measurement data. The coefficients of the control model including one or more of the group of the proportional coefficient A_{1,} the integral coefficient A₂, the inverse memory time scale λ, the steady depletion coefficient A₃, a feedback coefficient A₄, and F(t) which models food intake and circadian rhythm may be determined by fitting the representative curve to the proportional-integral controller equation, and the differential equation describing glucose response.

In one embodiment, use of the model allows for the determination of a metric based on one or more of A₁, A₂, A₃, A₄ and λ. In one embodiment, the metric is indicative of the effectiveness of the glucose homeostasis control system in a subject. In one embodiment, the metric is a digital biomarker of glucose homeostasis in the subject. In one embodiment, the metric is a dimensionless coefficient such as A₁/A₂. In one embodiment, the metric is based on the difference between A₂ and A₁ such as the metric R as described herein. In one embodiment, the metric is based on a measure of the distribution or variability of glucose measurements for the subject, optionally the standard distribution of some or all glucose measurements available for the subject. In one embodiment, the metric is based on one or more values of the control variable, optionally the maximum attained by the control variable such as in an optimal fit. In one embodiment, the metric is used to classify a subject into a glucose homeostasis phenotype, optionally into a prediabetic subphenotype.

In one embodiment, the method comprises comparing one or more metrics for a subject determined using the model described herein to one or more control metrics in order to evaluate glucose homeostasis in the subject relative to the one or more controls or to classify the subject into a glucose homeostasis phenotype. In one embodiment, the control metrics are representative of metrics determined for a population of subjects with glycemic dysfunction, such as subjects with type II diabetes. In one embodiment, the control is a threshold level indicative of a status of glycemic dysfunction in a group of subjects. For example, in one embodiment a male subject with a value of A₁ less than 0.25 is classified as having a weak instantaneous glucose response phenotype. In another embodiment, a female subject with a value of A₁ less than 0.30 is classified as having a weak instantaneous glucose response phenotype. In one embodiment, a subject with a value of A₂/λ less than 0.6 is classified as having a weak inertial glucose response phenotype.

Various devices known in the art can be used to produce time-series glucose data useful for generating a representative curve for a subject. For example, glucose levels can be gathered with off-the-shelf glucose monitoring devices such as continuous glucose monitoring (CGM) technology, which provides a convenient and cost-effective way to accurately measure continuous glycemia and provide glucose data suitable for generating representative curves for use in the systems and methods described herein.

As set out in the Example 1, glucose levels were monitored for 31 subjects over a period of 7-14 days using a commercially available CGM device. Representative curves were then generated for each subject and fit to the control model of glucose homeostasis thereby determining the coefficients of the control model (A₁, A₂, A₃, A₄ and λ). Notably, the control model was able to model each subject's representative curve with an average E-value of 0.018.

Analysis of the coefficients and/or metrics for each subject demonstrated inter-subject variability that, without being limited by theory, is expected to reflect glycemic function and homeostasis in the subject and help identify subject with glycemic dysfunction such as type 2 diabetes or pre-diabetes.

As set out in Example 3, analysis of coefficients and/or glucose homeostasis metrics was performed for a second cohort of subjects as well as an additional subject diagnosed with Type II diabetes. Notably, as shown in FIGS. 13-15, the diabetic subject exhibited a value for glucose homeostasis metric R that was readily distinguished from the values of R for those subjects without any known dysfunction in glucose homeostasis.

As shown in Example 4, glucose homeostasis was investigated in a group of ˜125 subjects including diabetics, prediabetics and normal individuals. Remarkably, prediabetic subjects were distinguished into different phenotypic clusters based on glucose homeostasis metrics A₁ and A₂/λ. These phenotypic clusters are associated with different underlying physiological states and represent different categories for disease progression and response to interventions such as the administration of therapeutics, frequency of monitoring or lifestyle changes.

Provided further are systems and methods for generating a glucose homeostasis model for a patient, and for providing screening, diagnostic, predictive, prognostic, and responsive messages to a user based on the glucose homeostasis model and the received glucose measurement data. In one embodiment, the messages provided to the user are indicative of a glucose homeostasis phenotype.

In a first aspect, some embodiments of the invention provide a method for generating a glucose homeostasis model for a subject, the method comprising: receiving, at a processor, a plurality of glucose measurements for the patient, the plurality of glucose measurements for the patient comprising a time-series collected from the patient using a glucose measurement device; selecting, at the processor, one or more curve intervals in the plurality of glucose measurements, the one or more curve intervals corresponding to one or more local maxima of the plurality of glucose measurements; determining, at the processor, a representative curve based on the one or more curve intervals; determining, at the processor, a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate; generating, at the processor, the glucose homeostasis model, the glucose homeostasis model comprising the proportional coefficient A₁, the integral coefficient A₂, the inverse memory time scale λ, the steady depletion coefficient A₃, and the feedback coefficient A₄.

In one or more embodiments, the determining, at the processor, the representative curve based on the one or more curve intervals may further comprise: normalizing, at the processor, the one or more curve intervals.

In one or more embodiments, the determining, at the processor, the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate may further comprise: determining, at the processor, a first approximate proportional coefficient, a first approximate integral coefficient and a first approximate inverse memory time scale of the representative curve based on an approximation of an integral of the representative curve; determining, at the processor, a first approximate steady depletion coefficient and a first approximate feedback coefficient based on a differential equation of the representative curve, the first approximate proportional coefficient, the first approximate integral coefficient, and the first approximate inverse memory time scale; and determining, at the processor, a first vector comprising the first approximate proportional coefficient, the first approximate integral coefficient, the first approximate inverse memory time scale, the first approximate steady depletion coefficient and the first approximate feedback coefficient.

In one or more embodiments, the determining, at the processor, the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate may further comprise: determining, at the processor, a second approximate proportional coefficient, a second approximate integral coefficient and a second approximate inverse memory time scale of the representative curve based on the approximation of an integral of the representative curve; determining, at the processor, a second approximate steady depletion coefficient and a second approximate feedback coefficient based on a differential equation of the representative curve, the second approximate proportional coefficient, the second approximate integral coefficient, and the second approximate inverse memory time scale; determining, at the processor, a second vector based on the second approximate proportional coefficient, the second approximate integral coefficient, the second approximate inverse memory time scale, the second approximate steady depletion coefficient and the second approximate feedback coefficient; comparing, at the processor, an error between the first vector and the second vector; and performing, at the processor, a gradient descent to modify the first approximate proportional coefficient, the first approximate integral coefficient, the first approximate inverse memory time scale, the first approximate steady depletion coefficient and the first approximate feedback coefficient.

In one or more embodiments, the determining, at the processor, the proportional coefficient A₁ for response of the controller u(t) to the error e(t), the integral coefficient A₂ for response of the controller u(t) to past values of error e(t), the inverse memory time scale λ for decay of an integral term, the steady depletion coefficient A₃ for the basic metabolic rate, and the feedback coefficient A₄ for the approximate mass action rate may further comprise: determining, at the processor, an input coefficient peak F*.

In one or more embodiments, the input coefficient peak F* may be determined using a Gaussian function.

In one or more embodiments, the determining, at the processor, the representative curve may further comprise: averaging, at the processor, the one or more normalized curve intervals; or averaging, at the processor, the one or more curve intervals to generate an average curve interval, and wherein the normalizing, at the processor, may comprise normalizing the average curve interval.

In one or more embodiments, the method may further comprise: determining, at the processor, a glucose homeostasis metric based on one or more of the group of the proportional coefficient A₁, the integral coefficient A₂, the steady depletion coefficient A₃, the feedback coefficient A₄, and the inverse memory time scale term λ; wherein the glucose homeostasis model may further comprise the glucose homeostasis metric. In one embodiment, the glucose homeostasis metric is based on one or more selected from the group consisting of the proportional coefficient A₁, the integral coefficient A₂ the inverse memory time scale term λ and combinations thereof. In one embodiment, the glucose homeostasis metric is A₁ or A₂/λ and is indicative of a glucose homeostasis phenotype for a subject.

In one or more embodiments, the method may further comprise determining, at the processor, a glucose homeostasis metric based on one or more of the proportional coefficient A₁, the integral coefficient A₂, glucose measurements for the subject, optionally a standard deviation of the glucose measurements, and an estimated value of the control variable u(t), optionally a maximum estimated value u(m). For example, in one embodiment the method comprises determining, at the processor, a glucose homeostasis metric R, the glucose homeostasis metric R based on the proportional coefficient A₁, the integral coefficient A₂, the standard deviation of glucose measurements for the subject σₑ, and the maximum attained by the control variable in the optimal fit uₘ. In one embodiment, the glucose homeostasis model further comprises the glucose homeostasis metric R.

In one embodiment, the glucose homeostasis metric R is determined as the product of the standard deviation of glucose measurements for the subject σₑ and the difference between the integral coefficient A₂ and the proportional coefficient A_{1,} divided by the maximum attained by the control variable in the optimal fit uₘ.

In one or more embodiments, the method may further comprise determining, at the processor, a glucose homeostasis metric B₁, the glucose homeostasis metric B₁ based on the proportional coefficient A₁, and the integral coefficient A₂, and the inverse memory time scale term λ; and wherein the glucose homeostasis model may further comprises the glucose homeostasis metric B₁.

In one or more embodiments, the glucose homeostasis metric B₁ may be determined as the product of the proportional coefficient A₁ and the inverse memory time scale term λ, divided by the integral coefficient A₂.

In one or more embodiments, the method may further comprise: determining, at the processor, a feedback loop metric B₂, the feedback loop metric B₂ based on the inverse memory time scale term λ and the feedback coefficient A₄; and wherein the glucose homeostasis model further comprises the feedback loop metric B₂.

In one or more embodiments, the feedback loop metric B₂ may be determined by dividing the inverse memory time scale term λ by the feedback coefficient A₄.

In one or more embodiments, the method may further comprise determining, at the processor, a glucose homeostasis metric associated with a glucose homeostasis phenotype such as A₁ or A₂/λ.

In one or more embodiments, the determining, at the processor, the first approximate proportional coefficient, the first approximate integral coefficient and the first approximate inverse memory time scale of the representative curve may be based on a midpoint rule approximation of the integral of the representative curve.

In one or more embodiments, the determining, at the processor, the first approximate steady depletion coefficient and the first approximate feedback coefficient may be based on applying Euler's method to the differential equation of the representative curve, the first approximate proportional coefficient, the first approximate integral coefficient, and the first approximate inverse memory time scale.

In one or more embodiments, the method may further comprise displaying, at a display device a glucose homeostasis metric. For example, in one embodiment, the glucose homestasis metric is at least one of the group of the glucose homeostasis metric R, the glucose homeostasis metric B₁, and the feedback loop metric B₂. In one embodiment, the glucose homeostasis metric is at least one of A₁ and A₂/λ.

In one or more embodiments, the method may further comprise: transmitting, at a network device, at least one of the group of a glucose homeostasis metric and the glucose homeostasis model to a remote service. In embodiment, the method comprises transmitting, at a network device, at least one of the glucose homestasis model, the glucose homeostasis metric R, the glucose homeostasis metric B₁, the glucose homeostasis metric A₁, the glucose homeostasis metric A₂/λ and the feedback loop metric B₂ to a remote service.

In one or more embodiments, the plurality of glucose measurements may be received from a glucose measurement device.

In one or more embodiments, the glucose measurement device may collect the plurality of glucose measurements at a configurable frequency.

In one or more embodiments, the glucose measurement device may be a FreeStyle™ Libre or another continuous glucose monitoring device.

In a second aspect, one or more embodiments provide a system for generating a glucose homeostasis model for a subject, the system comprising: a memory, the memory comprising a plurality of glucose measurements for the patient, the plurality of glucose measurements for the patient comprising a time-series collected from the patient using a glucose measurement device; a processor in communication with the memory, the processor configured to: select one or more curve intervals in the plurality of glucose measurements, the one or more curve intervals corresponding to one or more local maxima of the plurality of glucose measurements; determine a representative curve based on the one or more curve intervals; determine a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate; generate the glucose homeostasis model, the glucose homeostasis model comprising the proportional coefficient A₁, the integral coefficient A₂, the inverse memory time scale λ, the steady depletion coefficient A₃, and the feedback coefficient A₄. In one embodiment, the system is configured to classify the subject into a glucose homeostasis phenotype based on one or more glucose homeostasis metrics. In one or more embodiments, the processor may be further configured to determine the representative curve based on the one or more curve intervals by: normalizing the one or more curve intervals.

In one or more embodiments, the processor may be further configured to determine the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate by: determining a first approximate proportional coefficient, a first approximate integral coefficient and a first approximate inverse memory time scale of the representative curve based on an approximation of an integral of the representative curve; determining a first approximate steady depletion coefficient and a first approximate feedback coefficient based on a differential equation of the representative curve, the first approximate proportional coefficient, the first approximate integral coefficient, and the first approximate inverse memory time scale; and determining a first vector comprising the first approximate proportional coefficient, the first approximate integral coefficient, the first approximate inverse memory time scale, the first approximate steady depletion coefficient and the first approximate feedback coefficient.

In one or more embodiments, the processor may be further configured to determine the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate by: determining a second approximate proportional coefficient, a second approximate integral coefficient and a second approximate inverse memory time scale of the representative curve based on the approximation of an integral of the representative curve; determining a second approximate steady depletion coefficient and a second approximate feedback coefficient based on a differential equation of the representative curve, the second approximate proportional coefficient, the second approximate integral coefficient, and the second approximate inverse memory time scale; determining a second vector based on the second approximate proportional coefficient, the second approximate integral coefficient, the second approximate inverse memory time scale, the second approximate steady depletion coefficient and the second approximate feedback coefficient; comparing an error between the first vector and the second vector; and performing a gradient descent to modify the first approximate proportional coefficient, the first approximate integral coefficient, the first approximate inverse memory time scale, the first approximate steady depletion coefficient and the first approximate feedback coefficient.

In one or more embodiments, the processor may be further configured to determine the proportional coefficient A₁ for response of the controller u(t) to the error e(t), the integral coefficient A₂ for response of the controller u(t) to past values of error e(t), the inverse memory time scale λ for decay of an integral term, the steady depletion coefficient A₃ for the basic metabolic rate, and the feedback coefficient A₄ for the approximate mass action rate by: determining an input coefficient peak F*.

In one or more embodiments, the input coefficient peak F* may be determined using a Gaussian function.

In one or more embodiments, the processor may be further configured to determine the representative curve by: averaging the one or more normalized curve intervals; or averaging the one or more curve intervals to generate an average curve interval, and wherein the normalizing comprises normalizing the average curve interval.

In one or more embodiments, the processor may be further configured to: determine a glucose homeostasis metric based on one or more of the group of the proportional coefficient A₁, the integral coefficient A₂, the steady depletion coefficient A₃, the feedback coefficient A₄, and the inverse memory time scale term λ; wherein the glucose homeostasis model may further comprise the glucose homeostasis metric. In one embodiment, the glucose homeostasis metric is A₁ or A₂/λ.

In one or more embodiments, the processor may be further configured to determine a glucose homeostasis metric based on the proportional coefficient A₁, the integral coefficient A₂, a statistical measure of the glucose levels of the subject or their variation or distribution, such as a standard deviation, and an estimated value of the control variable u(t), such as an estimated maximal value. In one or more embodiments, the processor may be configured to determine a glucose homeostasis metric R, the glucose homeostasis metric R based on the proportional coefficient A₁, the integral coefficient A₂, the standard deviation of glucose measurements for the subject σₑ, and the maximum attained by the control variable in the optimal fit uₘ. For example in one embodiment the processor is configured to determine a glucose homeostasis metric

$$R = \frac{{σ}_{e} ( {A}_{2} - {A}_{1} )}{{u}_{m}} ) .$$

In one embodiment, the glucose homeostasis model further comprises the glucose homeostasis metric R.

In one or more embodiments, the processor may be further configured to: determine a glucose homeostasis metric B₁, the glucose homeostasis metric B₁ based on the proportional coefficient A₁, and the integral coefficient A₂, and the inverse memory time scale term λ; and wherein the glucose homeostasis model may further comprise the glucose homeostasis metric B₁.

In one or more embodiments, the glucose homeostasis metric B₁ may be determined as the product of the proportional coefficient A₁ and the inverse memory time scale term λ, divided by the integral coefficient A₂.

In one or more embodiments, the processor may be further configured to: determine a feedback loop metric B₂, the feedback loop metric B₂ based on the inverse memory time scale term 1 and the feedback coefficient A₄; and wherein the glucose homeostasis model may further comprise the feedback loop metric B_{2.}

In one or more embodiments, the feedback loop metric B₂ may be determined by dividing the inverse memory time scale term A by the feedback coefficient A₄.

In one or more embodiments, the processor may be further configured to determine the first approximate proportional coefficient, the first approximate integral coefficient and the first approximate inverse memory time scale of the representative curve is based on a midpoint rule approximation of the integral of the representative curve.

In one or more embodiments, the processor may be further configured to determine the first approximate steady depletion coefficient and the first approximate feedback coefficient based on applying Euler's method to the differential equation of the representative curve, the first approximate proportional coefficient, the first approximate integral coefficient, and the first approximate inverse memory time scale.

In one or more embodiments, the system may further comprise: a display device in communication with the processor. In one embodiment, the processor is further configured to display, at the display device, a glucose homeostasis metric. In one embodiment, the processor is configured to display, at the display device, at least one of the group of the glucose homeostasis metric R, the glucose homeostasis metric B₁, the glucose homeostasis metric A₁, the glucose homeostasis metric A₂/λ, and the feedback loop metric B₂. In another embodiment, the system may be configured to provide audio or haptic feedback to a user based on the glucose homeostasis metric.

In one or more embodiments, the system may further comprise: a network device in communication with the processor; and wherein the processor is further configured to: transmit, using the network device, a glucose homestasis model or a glucose homeostasis metric, to a remote service. For example, in one embodiment the processor is further configured to transmit, using the network device, at least one of the group of the glucose homeostasis model, the glucose homeostasis metric R, the glucose homeostasis metric B₁, the glucose homeostasis metric A₁, the glucose homeostasis metric A₂/λ and the feedback loop metric B₂ to a remote service.

In one or more embodiments, the system may further comprise a glucose measurement device in communication with the processor. In one embodiment, the plurality of glucose measurements may be received from the glucose measurement device.

In one or more embodiments, the glucose measurement device may collect the plurality of glucose measurements at a configurable frequency.

In one or more embodiments, the glucose measurement device may be a FreeStyle™ Libre, or another continuous glucose monitoring device.

In a third aspect, one or more embodiments provide a method for generating a glucose homeostasis message, the method comprising: receiving, at a processor, a glucose homeostasis model, the glucose homeostasis model comprising a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate; receiving, at a processor, one or more current glucose measurements; determining, at the processor, a glucose message based on the glucose homeostasis model, and the one or more current glucose measurements; and displaying, at a display device, the glucose homeostasis message. In one embodiment, the glucose homeostasis message is a glucose homeostasis phenotype. In one embodiment, the subject is prediabetic and the glucoses homeostasis phenotype is a prediabetic subphenotype.

In one or more embodiments, the determining, at the processor, the glucose message based on the glucose homeostasis model, and the one or more current glucose measurements may further comprise: determining, at the processor, a glucose screening message, the glucose screening message for predicting a likelihood that a user has a health condition; and wherein the glucose homeostasis message may be the glucose screening message.

In one or more embodiments, the glucose message may be a percentage chance of the health condition, and the health condition is type 2 diabetes.

In one or more embodiments, the determining, at the processor, the glucose message based on the glucose homeostasis model, and the one or more current glucose measurements may further comprise: determining, at the processor, a glucose diagnostic message, the glucose diagnostic message for a glucose diagnostic measurement; and wherein the glucose homeostasis message may be the glucose diagnostic message.

In one or more embodiments, the determining, at the processor, the glucose message based on the glucose homeostasis model, and the one or more current glucose measurements may further comprise: determining, at the processor, a glucose predictive message, the glucose predictive message for predicting that a user will develop a health condition; and wherein the glucose homeostasis message may be the glucose predictive message.

In one or more embodiments, the determining, at the processor, the glucose message based on the glucose homeostasis model, and the one or more current glucose measurements may further comprise: determining, at the processor, a glucose prognostic message, the glucose prognostic message for predicting whether a health condition of a user is more likely to respond to an intervention; and wherein the glucose homeostasis message may be the glucose prognostic message.

In one or more embodiments, the determining, at the processor, the glucose message based on the glucose homeostasis model, and the one or more current glucose measurements may further comprise: determining, at the processor, a glucose response message, the glucose response message for predicting a performance of a current intervention; wherein the glucose homeostasis message may be the glucose response message.

In one or more embodiments, the determining, at the processor, the glucose message based on the glucose homeostasis model and the one or more current glucose measurements comprises determining, at the processor, a glucose homeostasis metric as described herein. For example, in one embodiment the glucose homeostasis metric is based on one or more of the group of the proportional coefficient A₁, the integral coefficient A₂, the steady depletion coefficient A₃, the feedback coefficient A₄, and the inverse memory time scale term λ. In one embodiment, the method optionally comprise comparing the glucose homeostasis metric to a control. In one embodiment, the glucose homeostasis metric is R. In one embodiment, the glucose homeostasis metric is useful for classifying the subject into a glucose homeostasis phenotype such as A₁ or A₂/λ.

In a fourth aspect, one or more embodiments provide a system for generating a glucose homeostasis message, the system comprising: a memory, the memory comprising: a glucose homeostasis model, the glucose homeostasis model comprising: a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate; a display device; a processor in communication with the memory and the display device, the processor configured to: receive one or more current glucose measurements; determine a glucose message based on the glucose homeostasis model, and the one or more current glucose measurements; and displaying, at the display device, the glucose homeostasis message.

In one or more embodiments, the processor may be further configured to determine the glucose message based on the glucose homeostasis model, and the one or more current glucose measurements by: determining a glucose screening message, the glucose screening message for predicting a likelihood that a user has a health condition; and wherein the glucose homeostasis message may be the glucose screening message.

In one or more embodiments, the glucose message may be a glucose homeostasis phenotype.

In one or more embodiments, the glucose message may be a percentage chance of the health condition. In one embodiment, the health condition is type 2 diabetes. In another embodiment, the health condition is type 1 diabetes. In another embodiment, the health condition is pre-diabetes and/or a prediabetic subphenotype.

In one or more embodiments, the processor may be further configured to determine the glucose message based on the glucose homeostasis model, and the one or more current glucose measurements by: determining a glucose diagnostic message, the glucose diagnostic message for a glucose diagnostic measurement; and wherein the glucose homeostasis message may be the glucose diagnostic message.

In one or more embodiments, the processor may be further configured to determine the glucose message based on the glucose homeostasis model, and the one or more current glucose measurements by: determining a glucose predictive message, the glucose predictive message for predicting that a user will develop a health condition; and wherein the glucose homeostasis message may be the glucose predictive message.

In one or more embodiments, the processor may be further configured to determine the glucose message based on the glucose homeostasis model, and the one or more current glucose measurements by: determining a glucose prognostic message, the glucose prognostic message for predicting whether a health condition of a user is more likely to respond to an intervention; and wherein the glucose homeostasis message may be the glucose prognostic message.

In one embodiment, the processor is configured to determine, at the processor, a glucose homeostasis metric and optionally compare the glucose homestasis metric to a control. In one embodiment, the glucose homeostasis metric is based on one or more of the group of the proportional coefficient A₁, the integral coefficient A₂, the steady depletion coefficient A₃, the feedback coefficient A₄, and the inverse memory time scale term λ. In one embodiment, the glucose homeostasis metric is R.

### BRIEF DESCRIPTION OF THE DRAWINGS

A preferred embodiment of the present invention will now be described in detail with reference to the drawings, in which:

FIG. 1 shows one embodiment of a system diagram of a digital biomarker system for evaluating glucose homeostasis.

FIG. 2 shows a block diagram of the mobile device from FIG. 1.

FIG. 3 shows one embodiment of a software component diagram of the glucose monitoring device from FIG. 1.

FIG. 4A shows an example of glucose time series data.

FIG. 4B shows an analysis function including a derivative and integral function of the glucose time series data in FIG. 4A.

FIG. 5 shows another example glucose time series data.

FIG. 6A shows an example of glucose time series data having overlaid sample peaks.

FIG. 6B shows a representative peak of the glucose time series data in FIG. 6A.

FIG. 7 shows an example proportional-integral model.

FIG. 8A shows an example method for determining a glucose control model.

FIG. 8B shows another example method for determining a glucose

control model.

FIG. 8C shows an example method for using the glucose control model.

FIGS. 9A-F shows measured and model values for a glucose time series for 6 different subjects including plotted values for the glucose controller function (u) and food source (F(t)).

FIG. 10A shows the grouping of B-values for study participants.

FIG. 10B shows a plot of B-values vs. E-values for study participants

FIGS. 11A-11F show drawings of various embodiments of a user interface.

FIG. 12 shows a distribution diagram 1200 of the indicator R.

FIG. 13 shows the optimal model parameters for all subjects with A₂ (y-axis) vs. A₁ (x-axis) including the original data (Example 1) as well as the MGCTS data and pilot diabetic trial (Example 3).

FIG. 14 shows a histogram of the glucose homeostasis marker B (B=A₁/A₂).

FIG. 15 shows a histogram of the glucose homeostasis marker R

$$( R = \frac{{σ}_{e} ( {A}_{2} - {A}_{1} )}{{u}_{m}} ) .$$

FIG. 16 shows clustering of male and female prediabetic subjects based on values of A₁.

FIG. 17 shows clustering of male and female prediabetic subjects based on values of A₂/λ.

FIG. 18 shows a plot of A₂/λ vs. A₁ for prediabetic female subjects (FIG. 18A) and prediabetic male subjects (FIG. 18B).

FIG. 19 shows a plot of A₂/λ vs. A₁ for all female subjects (FIG. 19A) and all male subjects (FIG. 19B) including prediabetic, normal and diabetic subjects.

FIG. 20 shows a plot of A₂/λ vs. A₁ for female prediabetic subjects including the identification of three female prediabetic subphenotypes.

FIG. 21 shows a plot of A₂/λ vs. A₁ for male prediabetic subjects including the identification of four male prediabetic subphenotypes.

FIG. 22 shows a plot of A₂/λ vs. A₁ for female subjects (see FIG. 19A—female subjects) including female gestational study subjects.

### DESCRIPTION OF EXEMPLARY EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

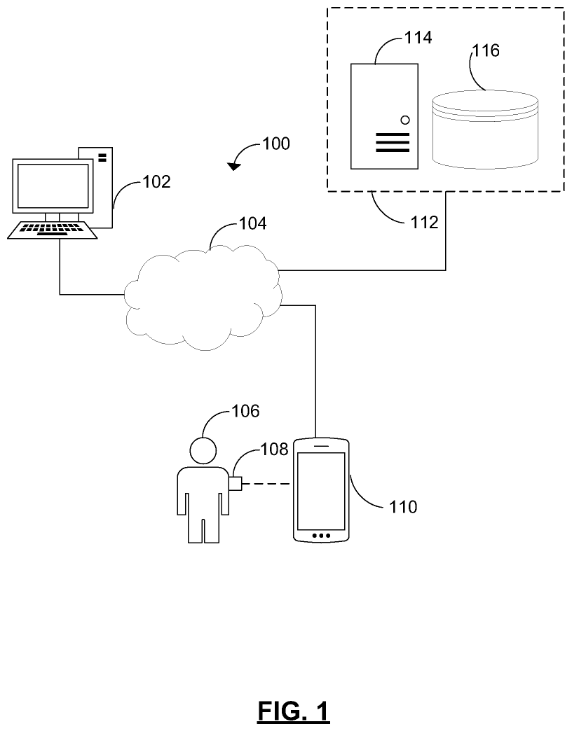

Reference is first made to FIG. 1, there is shown a system diagram 100 of a digital biomarker system for evaluating glucose homeostasis. The digital biomarker system includes one or more user devices 102, a network 104, a user 106, a glucose monitoring device 108, a mobile device 110, and a remote service 112.

The one or more user devices 102 may be used by an end user to access a software application (not shown) running on processing server 114 at remote service 112 over network 104. For example, the application may be a web application, or a client/server application. The user device 102 may be a desktop computer, mobile device, or laptop computer. The user device 102 may be in communication with processing server 114, and may allow a user to review a user profile stored in database 116. The user 106 at user device 102 may also be an administrator user who may administer the configuration of the digital biomarker system using a web application at processing server 114.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

User 106 may be a patient using a glucose monitoring device 108, or an individual who uses a glucose monitoring device 108 for informational purposes. The user 106 may create a user profile on remote service 112 that may remotely track the glucose measurement data, glucose homeostasis model data, determined metrics, or other user information. The systems and methods described herein may also be used by a health professional, such as a doctor or nurse or dietician, for evaluating or consulting a patient.

Glucose measurement device 108 may measure the glucose levels of the user. The glucose levels may be measured based on blood glucose levels, or interstitial glucose levels. The glucose measurement device 108 may measure real-time glucose data for the user. The glucose measurement device 108 may measure continuous interstitial glucose levels. The glucose measurement device 108 may measure glucose data using a flexible filament inserted through the skin into the user's body. The glucose measurement device 108 may measure glucose data based on the glucose-oxidase process and may measure an electrical current proportional to the concentration of glucose. The glucose measurement device 108 may contain a sensor which is attached to the user with an adhesive patch, optionally to a posterior region of the upper arm of the user. The glucose measurement device may further include an optional handheld reader device (not shown) which communicates with the sensor via near-field communication. Glucose concentrations (e.g. in mmol/L) may be captured by the sensor at regular or irregular time intervals (e.g. every 15 min) and/or when users scan the sensor using the optional handheld device. The data capture frequency of the sensor of the glucose measurement device 108 may be configurable, for example the data capture may occur at different measurement frequencies such as every 10 min, 5 min, every 2 minutes etc. In one embodiment, the data capture by the sensor may occur at least 3, 4, 5, 6, 7, 8, 9, 10, 11, 12, 13, 14, 15, 16, 17, 18, 19, or 20 times per hour. In another embodiment, high-frequency data capture by the sensor may occur at least 30, 40, 50, 60, or 120 times per hour.

The glucose data may be captured wirelessly by the handheld device associated with the glucose monitoring device 108, using a wired connection to the handheld device associated with the glucose monitoring device 108, wirelessly by the mobile device 110, or using a wired connection to the mobile device 110. The handheld device of the glucose measurement device 108 may be scanned at regular intervals to transfer glucose data, such as every 8 hours. The glucose measurement device 108 may have a replaceable sensor, for example the sensor may be replaced approximately every 14 consecutive days.

In one embodiment, the glucose measurement device 108 is a continuous glucose monitor (CGM) device that directly or indirectly provides a measure of glucose concentration. Various CGM devices known in the art are suitable for use with the systems and methods described herein. In one embodiment, the glucose measurement device 108 may be the FreeStyle Libre™ glucose monitoring system available from Abbott® Diabetes Care. In another embodiment, the glucose measurement device 108 may be a CGM device from Dexcom (San Diego, California) such as the G6™, or a CGM device from Medtronic (Fridley, Minnesota) such as the Guardian™ Connect.

In a preferred embodiment, the functions of the optional handheld device of the glucose monitoring device may be performed by the mobile device 110. In this embodiment, the glucose tracking application on the mobile device 110 may communicate with the sensor and may download the glucose measurement data itself. The sensor of the glucose monitoring device may communicate with the mobile device 110 and the glucose tracking application using a local wireless connection, such as 802.11x, Bluetooth, Near-Field Communications (NFC), or Radio-Frequency IDentification (RFID).

The glucose measurement data collected by the glucose monitoring device 108 may include a glucose concentration, a time reference, glucose monitoring device information corresponding to the glucose monitoring device, and glucose measurement metadata.

The mobile device 110 may be any two-way communication device with capabilities to communicate with other devices. A user device 110 may be a mobile device such as mobile devices running the Google® Android® operating system or Apple® iOS® operating system.

Each user device 110 includes and executes a client application, such as a glucose tracking application, to communicate with the glucose monitoring device 108. The glucose tracking application may be a web application provided by server 114 of remote service 112, or it may be an application installed on the user device 110, for example, via an app store such as Google® Play® or the Apple® App Store®

The glucose tracking application on mobile device 110 may communicate with remote service 112 using an Application Programming Interface (API) endpoint, and may send and receive glucose measurement data, glucose homeostasis model data, user data, mobile device data, mobile device metadata, and determined metrics.

The glucose tracking application on mobile device 110 may communicate with the glucose measurement device 108 using a local wireless connection, such as an 802.11x connection, a Bluetooth connection, or other local wireless connection standards as are known.

In an alternate embodiment, the glucose measurement device 108 may communicate with the remote service 112, and may send and receive glucose measurement data, glucose homeostasis model data, user data, mobile device data, mobile device metadata, and/or determined metrics.

The remote service 112 is in network communication with the mobile device 110 and the one or more user devices 102. The remote service 112 may have a processing server 114 and a database 116. The database 116 and the processing server 114 may be provided on the same server, may be configured as virtual machines, or may be configured as containers. The remote server 112 may run on a cloud provider such as Amazon® Web Services (AWS®).

In an alternate embodiment, the remote service 112 may be in network communication with the glucose measurement device 108 directly.

The processing server 114 may host a web application or an Application Programming Interface (API) endpoint that the mobile device 110 or glucose measurement device 108 may interact with via network 104. The processing server 114 may make calls to the mobile device 110 to poll for glucose measurement data. Further, the processing server 114 may make calls to the database 116 to query patient data, glucose measurement data, glucose homeostasis model data, or other determined metrics. The requests made to the API endpoint of processing server 114 may be made in a variety of different formats, such as JavaScript Object Notation (JSON) or extensible Markup Language (XML).

The database 116 may store patient information including glucose measurement data history, user information including user profile information, glucose measurement device information, and configuration information. The database 116 may be a Structured Query Language (SQL) such as PostgreSQL or MySQL or a not only SQL (NoSQL) database such as MongoDB.

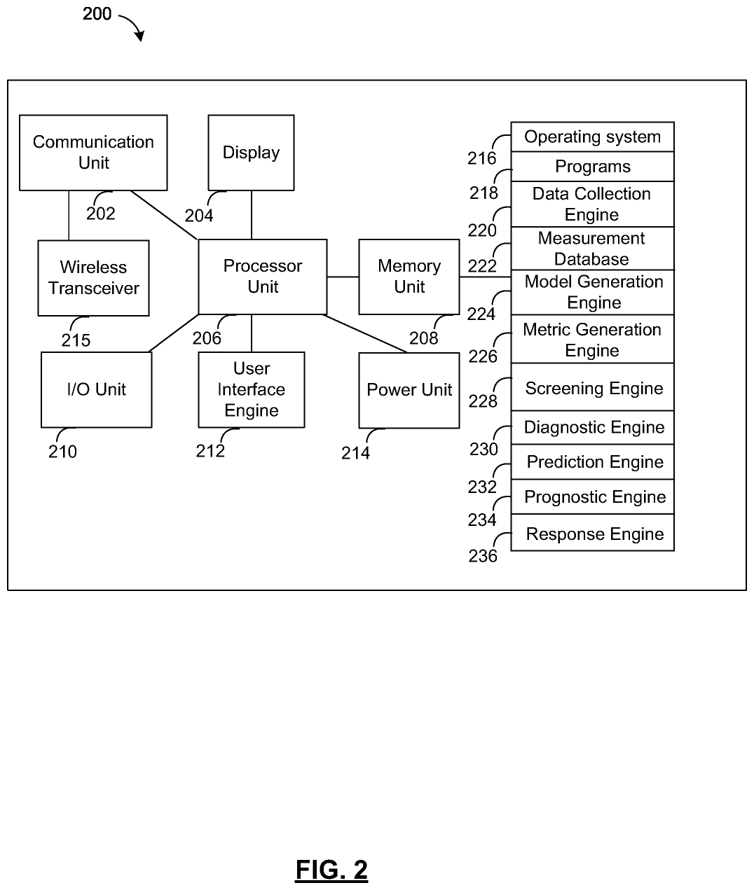

Reference is next made to FIG. 2, there is shown a block diagram 200 of the mobile device 110 from FIG. 1. As noted above, the mobile device 110 may wirelessly communicate with a sensor of the glucose measurement device 108 (see e.g. FIG. 1). Alternatively, mobile device 110 may communicate with glucose measurement device 108 through a wired connection.

The mobile device 200 includes one or more of a communication unit 202, a display 204, a processor unit 206, a memory unit 208, I/O unit 210, a user interface engine 212, a power unit 214, and a wireless transceiver 215.

The communication unit 202 can include wired or wireless connection capabilities. The communication unit 202 can include a radio that communicates utilizing CDMA, GSM, GPRS or Bluetooth protocol according to standards such as IEEE 802.11a, 802.11b, 802.11g, or 802.11n. The communication unit 202 can be used by the mobile device 200 to communicate with other devices or computers.

Communication unit 202 may communicate with the wireless transceiver 215 to transmit and receive information via local wireless network with the sensor of the glucose monitoring device. In an alternate embodiment, the communication unit 202 may communicate with the wireless transceiver 215 to transmit and receive information via local wireless network with the optional handheld device of the glucose monitoring device. The communication unit 202 may provide communications over the local wireless network using a protocol such as Bluetooth (BT) or Bluetooth Low Energy (BLE).

The display 204 may be an LED or LCD based display, and may be a touch sensitive user input device that supports gestures.

The processor unit 206 controls the operation of the mobile device 200. The processor unit 206 can be any suitable processor, controller or digital signal processor that can provide sufficient processing power depending on the configuration, purposes and requirements of the user device 200 as is known by those skilled in the art. For example, the processor unit 206 may be a high performance general processor. In alternative embodiments, the processor unit 206 can include more than one processor with each processor being configured to perform different dedicated tasks. In alternative embodiments, it may be possible to use specialized hardware to provide some of the functions provided by the processor unit 206. For example, the processor unit 206 may include a standard processor, such as an Intel® processor, an ARM® processor or a microcontroller.

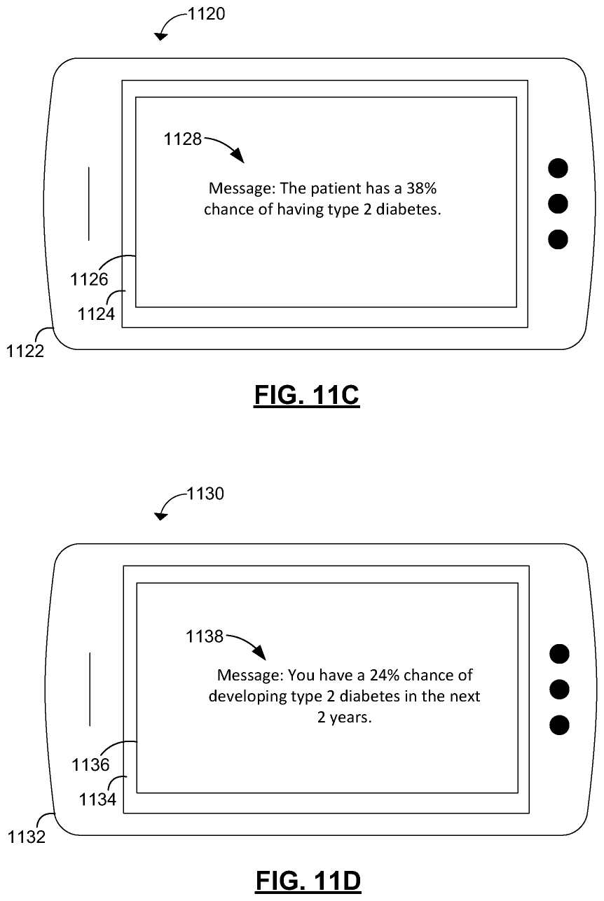

The processor unit 206 can also execute a user interface (UI) engine 212 that is used to generate various UIs, some examples of which are shown and described herein, such as interfaces shown in FIG. 11A, FIG. 11B, FIG. 11C, FIG. 11D, FIG. 11E, and FIG. 11F.

The memory unit 208 comprises software code for implementing an operating system 216, programs 218, data collection engine 220, measurement database 222, model generation engine 224, and optionally one or more of metric generation engine 226, screening engine 228, diagnostic engine 230, prediction engine 232, prognostic engine 234, and response engine 236.

The memory unit 208 can include RAM, ROM, one or more hard drives, one or more flash drives or some other suitable data storage elements such as disk drives, etc. The memory unit 208 is used to store an operating system 216 and programs 218 as is commonly known by those skilled in the art.

The I/O unit 210 can include at least one of a mouse, a keyboard, a touch screen, a thumbwheel, a track-pad, a track-ball, a card-reader, voice recognition software and the like again depending on the particular implementation of the user device 200. In some cases, some of these components can be integrated with one another.

The user interface engine 212 is configured to generate interfaces for users to configure glucose measurement, connect to the glucose measurement device, view glucose measurement data, view glucose metrics, view glucose screening messages, view glucose diagnostic messages, view glucose prediction messages, view glucose prognostic messages, and/or view glucose response messages. The various interfaces generated by the user interface engine 212 are displayed to the user on display 204. In some embodiments, the user interface may be configured to provide audio or haptic feedback to a user.

The power unit 214 can be any suitable power source that provides power to the user device 200 such as a power adaptor or a rechargeable battery pack depending on the implementation of the user device 200 as is known by those skilled in the art.

The operating system 216 may provide various basic operational processes for the user device 200. For example, the operating system 216 may be a mobile operating system such as Google® Android® operating system, or Apple® iOS® operating system, or another operating system.

The programs 218 include various user programs so that a user can interact with the user device 200 to perform various functions such as, but not limited to, viewing glucose data, metrics, as well as receiving messages as the case may be.

The data collection engine 220 receives glucose measurement data from the glucose measurement device (see e.g. 108 in FIG. 1) via the wireless transceiver 215 and the communication unit 202. The data collection engine 220 may receive the glucose measurement data and may store it in measurement database 222. The data collection engine 220 may supplement the glucose measurement data that is received from the glucose measurement device (see e.g. 108 in FIG. 1) with mobile device data and mobile device metadata. The data collection engine 220 may further send glucose measurement data to the remote service (see e.g. 112 in FIG. 1). The data collection engine 220 may communicate with the glucose measurement device wirelessly, using a wired connection, or using a computer readable media such as a flash drive or removable storage device.

The measurement database 222 may be a database for storing glucose measurement data from the glucose measurement device. The measurement database 222 may receive the data from the data collection engine 220, and may further receive queries for information from the model generation engine 224, the metric generation engine 226, the screening engine 228, the diagnostic engine 230, the prediction engine 232, the prognostic engine 234 and the response engine 236.

The measurement database 222 may be a database for storing models generated by the model generation engine 224, metrics generated by the metric generation engine 226, screening messages generated by the screening engine 228, diagnostic messages generated by the diagnostic engine 230, prediction messages generated by the prediction engine 232, prognostic messages generated by the prognostic engine 234, and/or response messages generated by the response engine 236.

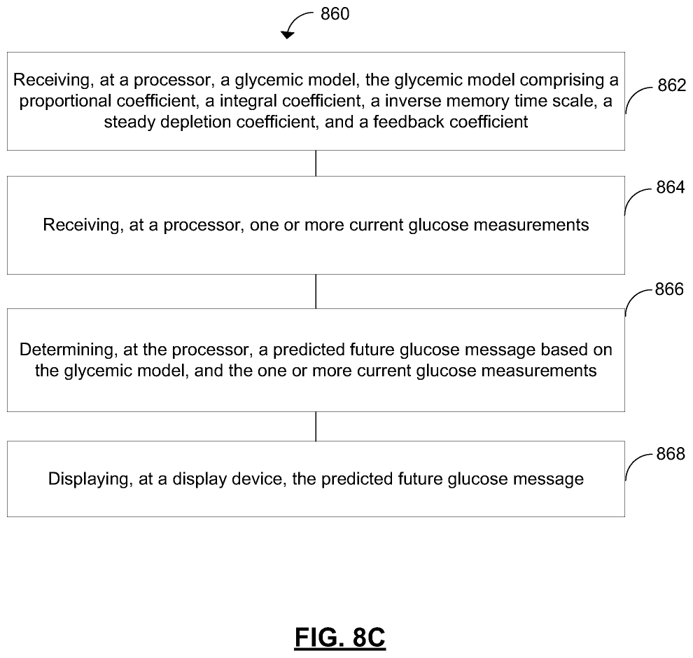

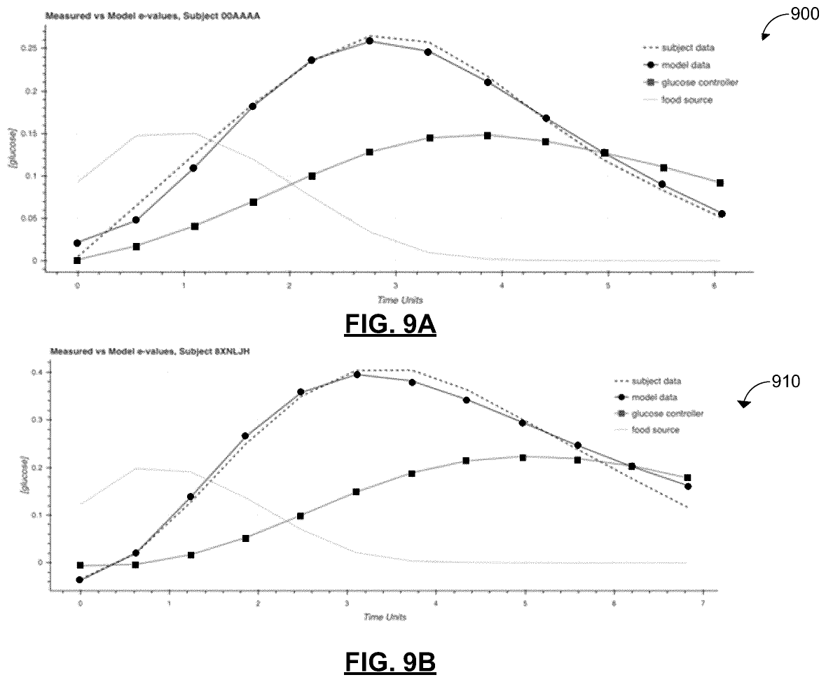

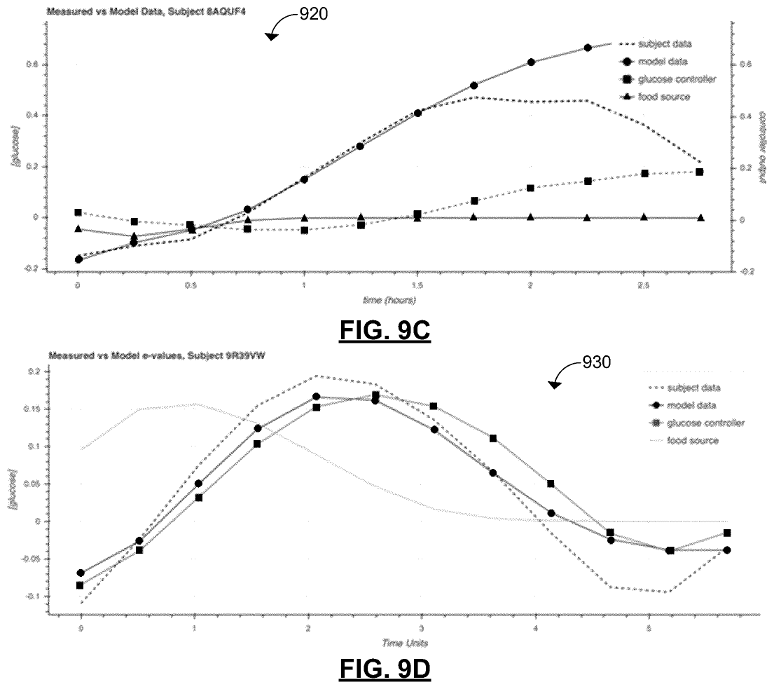

The model generation engine 224 may determine, based on glucose measurement data, a model including coefficients that describes the glycemic function of a user. For example, the model generation engine 224 may apply the method of FIG. 8A and FIG. 8B to determine A₁, A₂, A₃, A₄, and A coefficients as described herein.

The metric generation engine 226 may determine one or more metrics, based on glucose measurement data, and/or the glucose homeostasis model generated by the model generation engine 224. For example, the metric generation engine 226 may determine metrics based on one or more of the A₁, A₂, A₃, A₄, and λ coefficients as described herein. In one embodiment, the metric generation engine determines one or more of the R, B₁, and B₂ metrics as described herein. In one embodiment, the metric generation engine determines one or more metrics associated with classifying a subject into a glucose homeostasis phenotype, optionally A₁ or A₂/λ as described herein.

The screening engine 228 may determine screening messages based on the glucose homeostasis model generated by the model generation engine 224 and the glucose measurement data. The screening messages may be displayed to a user of the mobile device 200 using display 204. The screening messages may include a determination of a glucose homeostasis phenotype, optionally a prediabetic subphenotype. The screening messages may include a determination suggesting that a user is at a higher likelihood of having a health condition. For example, the screening message may include a percentage value of the risk of the health condition for a user over the general population.

Diagnostic engine 230 may determine diagnostic messages based on the glucose homeostasis model generated by the model generation engine 224 and the glucose measurement data. The diagnostic messages may be displayed or otherwise communicated to a user of the mobile device 200 using display 204. The diagnostic messages may include a determination suggestion that may substitute or augment for a healthcare professional confirming the presence of an underlying health condition. For example, the diagnostic message may include a diagnostic determination of the health condition. For example, the diagnostic message may indicate a continuous and/or history of glucose levels in a patient.

Prediction engine 232 may determine predictive messages based on the glucose homeostasis model generated by the model generation engine 224 and the glucose measurement data. The predictive messages may be displayed to a user of the mobile device 200 using display 204. The predictive messages may include a determination that suggests a user is likely to develop a health condition that they do not currently have (or isn't manifested sufficiently to be diagnosed easily) compared to the general population. For example, the predictive message may include a prediction that a non-diabetic individual will develop type 2 diabetes. In an alternate example, the predictive message may predict the user's glucose levels in the future. In one embodiment, the predictive message may predict a subject's response to treatment based on a glucose homeostasis phenotype.

Prognostic engine 234 may determine prognostic messages based on the glucose homeostasis model generated by the model generation engine 224 and the glucose measurement data. The prognostic messages may be displayed to a user of the mobile device 200 using display 204. The prognostic messages may include a determination that suggests a person with a known health condition is more likely to respond to a particular intervention than the general population. For example, the prognostic message may include a likelihood that the user may respond to an exercise regimen in order to reduce their risk of a health condition. The prognostic message may include a determination that a person with prediabetes is more likely to respond to a particular intervention than the general population based on the subject's glucose homeostasis phenotype, optionally based on a prediabetic subphenotype.

Response engine 236 may determine response messages based on the glucose homeostasis model generated by the model generation engine 224 and the glucose measurement data. The response messages may be displayed to a user of the mobile device 200 using display 204. The response messages may include a determination that suggests that an intervention currently underway by the user is working to treat a condition. For example, the response message may include a likelihood that the user's intervention to participate in an exercise regimen is working to reduce their risk of a health condition. In one embodiment, the response message may include a likelihood that the administration of a particular treatment, such as metformin, is working to reduce the risk of a health condition or a worsening health condition.

In the preferred embodiment, the functions of the data collection engine 220, measurement database 222, model generation engine 224, metric generation engine 226, screening engine 228, diagnostic engine 230, prediction engine 232, prognostic engine 234, and/or response engine 236 may be performed by the mobile device (see e.g. 110 in FIG. 1).

In an alternate embodiment, some or all of the functions of the data collection engine 220, measurement database 222, model generation engine 224, metric generation engine 226, screening engine 228, diagnostic engine 230, prediction engine 232, prognostic engine 234, and/or response engine 236 may be performed by an optional handheld device (not shown) of the glucose monitoring device.

In an alternate embodiment, some or all of the functions of the data collection engine 220, measurement database 222, model generation engine 224, metric generation engine 226, screening engine 228, diagnostic engine 230, prediction engine 232, prognostic engine 234, and/or response engine 236 may be performed by the remote service (see e.g. 112 in FIG. 1) of the glucose monitoring system.

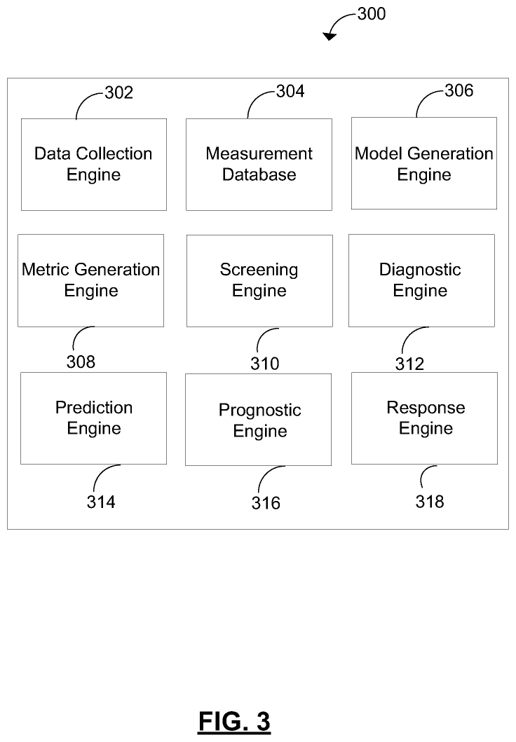

Reference is next made to FIG. 3, there is shown a software component diagram 300 of the mobile device 110 from FIG. 1. The software components include the data collection engine 302, the measurement database 304, the model generation engine 306, the metric generation engine 308, the screening engine 310, the diagnostic engine 312, the prediction engine 314, the prognostic engine 316, and the response engine 318.

The data collection engine 302 functions to receive glucose measurement data, and prepare the measurement data for the measurement database. The data collection engine 302 may include a processing queue for storing received glucose measurement data temporarily.

The measurement database 304 functions to store the glucose measurement data, and other data as described herein.

The model generation engine 306 functions to determine a glucose homeostasis model for a user. The glucose homeostasis model may include the A₁, A₂, A₃, A₄, and λ coefficients as described herein. The model generation engine 306 may function to determine a model for a Proportional-Integral control. The model generation engine 306 may apply an area under the curve approximation on the glucose measurement data. The area under the curve approximation may be an algorithmic implementation of the midpoint rule. The model generation engine 306 may determine a solution for a differential equation based on a known differential equation.

The metric generation engine 308, functions to determine metrics for a user based on the glucose homeostasis model for a user generated by the model generation engine 306. For example, the generated metrics may include the R, B₁ and B₂ metrics or another metric as described herein. In one embodiment, the metric is a digital biomarker indicative of glycemic control or glucose homeostasis in the subject. In one embodiment, the metric is a digital biomarker indicative of a glucose homeostasis phenotype based on nee or more of the A₁, A₂, A₃, A₄, and λ coefficients as described herein, optionally A₁ or A₂/λ. In one embodiment, the methods and systems described herein further comprise treating or monitoring the subject based on the classification of the subject into a glucose homeostasis phenotype, optionally based on a prediabetic subphenotype.

In one embodiment, one or more metrics determined for a subject may be compared to one or more control metrics representative of subjects with pre-determined diagnostic, prognostic, predictive or responsive criteria. In one embodiment, the control metrics are pre-determined values, optionally based on a plurality of control subjects. For example, in one embodiment the control metrics are representative of subjects with type 2 diabetes and similarity between the control metric and the subject metric is indicative of type 2 diabetes in the subject. In one embodiment, the control metric is a threshold value and a subject metric above or below the threshold is indicative of a pre-determined outcome or dysfunction associated with the threshold In one embodiment the control metric is a threshold associated with a glucose homeostasis phenotype.

For example, in one embodiment, the subject is a male subject and a subject with a value of A₁ less than 0.25 is classified as having a poor instantaneous glucose response phenotype. In one embodiment, the subject is a female subject and a subject with a value A₁ less than 0.30 is classified as having a poor instantaneous glucose response phenotype. Alternatively, in one embodiment the subject is a male subject and a subject with a value of A₁ greater than 0.25 is classified as having a strong instantaneous glucose response phenotype. In one embodiment, the subject is a female subject and a subject with a value A₁ greater than 0.30 is classified as having a strong instantaneous glucose response phenotype. In one embodiment, a subject with a value of A₂/λ less than 0.6 is classified as having a poor inertial glucose response phenotype. In one embodiment, a subject with a value of A₂/λ greater than 0.6 is classified as having a strong inertial glucose response phenotype.

In one embodiment, a female subject with a value of A₁ less than 0.3 and a value of A₂/λ greater than 0.6 is classified as having a first prediabetic subphenotype, a female subject with a value of A₁ less than 0.3 and a value of A₂/λ less than 0.6 is classified as having a second prediabetic subphenotype and a female subject with a value of A₁ greater than 0.3 and a value of A₂/λ less than 0.6 is classified as having a third prediabetic subphenotype. In one embodiment, a female subject with the first prediabetic subphenotype is selected for treatment to improve insulin sensitivity, optionally for treatment with metformin.

In one embodiment, a male subject with a value of A₁ less than 0.25 and a value of A₂/λ greater than 0.6 is classified as having a first prediabetic subphenotype, a male subject with a value of A₁ less than 0.25 and a value of A₂/λ less than 0.6 is classified as having a second prediabetic subphenotype, a male subject with a value of A₁ greater than 0.3 and a value of A₂/λ less than 0.6 is classified as having a third prediabetic subphenotype, and a male subject with a value of A₁ greater than 0.3 and a value of A₂/λ greater than 0.6 is classified as having a fourth prediabetic subphenotype. In one embodiment, a male subject with the first prediabetic subphenotype is selected for treatment to improve insulin sensitivity, optionally for treatment with metformin.

The screening engine 310 may generate screening messages.

The diagnostic engine 312 may generate diagnostic messages.

The prediction engine 314 may generate prediction messages.

The prognostic engine 316 may generate prognostic messages.

The response engine 318 may generate response messages.

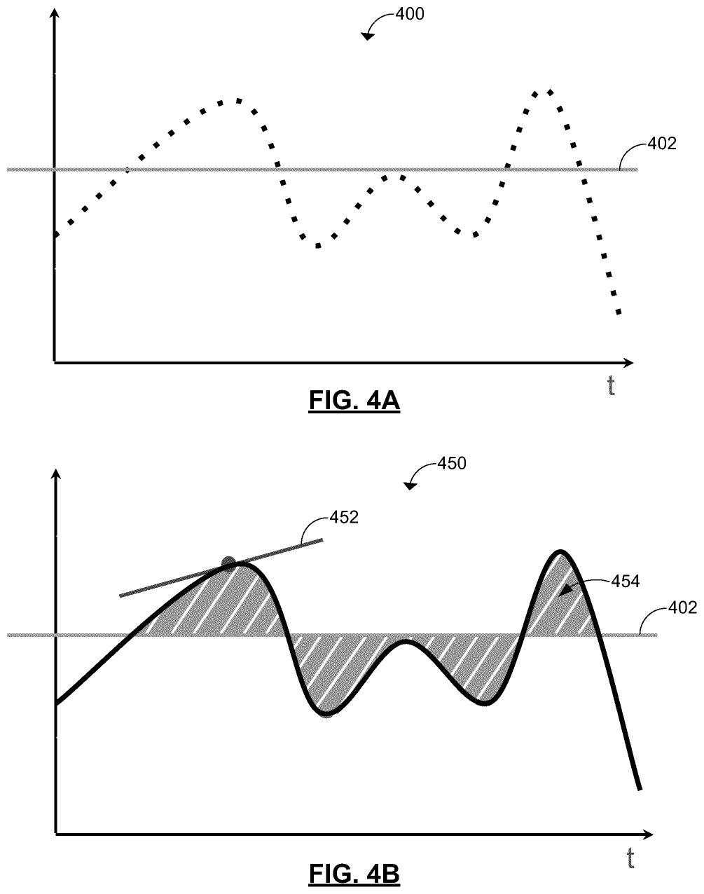

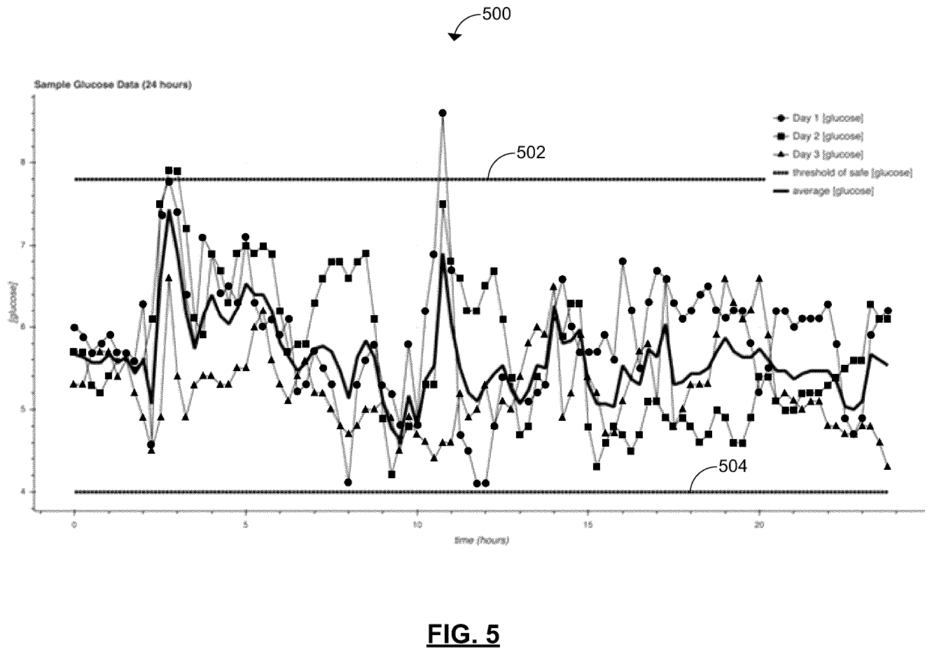

Reference is next made to FIG. 4A, there is shown an example diagram 400 of glucose time series data. Glucose levels in a user may be collected using a continuous glucose monitoring (CGM) device such as the glucose monitoring device (see 108 in FIG. 1), which provide for accurate and continuous glucose measurements. The example diagram 400 shows an example glucose time series, including data points that may be recorded over a period of time for a user and a set point 402 representing a target for glucose homeostasis of a user. The frequency of glucose data collection by the glucose monitoring device may be configurable. In one embodiment, the frequency of glucose data capture by the glucose monitoring device is at least 3, 4, 5, 6, 7, 8, 9, 10, 11 or 12 discrete measurements per hour. For example, in one embodiment, glucose levels are captured by the glucose monitoring device every 20 minutes, every 15 minutes, every 10 minutes, every 5 minutes, or every one minute.

Reference is next made to FIG. 4B, there is shown an analysis function 450 including a derivative function 452 and integral function 454 of the example diagram of glucose time series data in FIG. 4A. The derivative function 452 may be determined as a generally instantaneous rate of change of measured glucose levels. The integral function 454 may be determined as the area under the curve bounded by a set point 402, and may represent a term reflecting the prior history of the glucose measurement data around the set point 402.

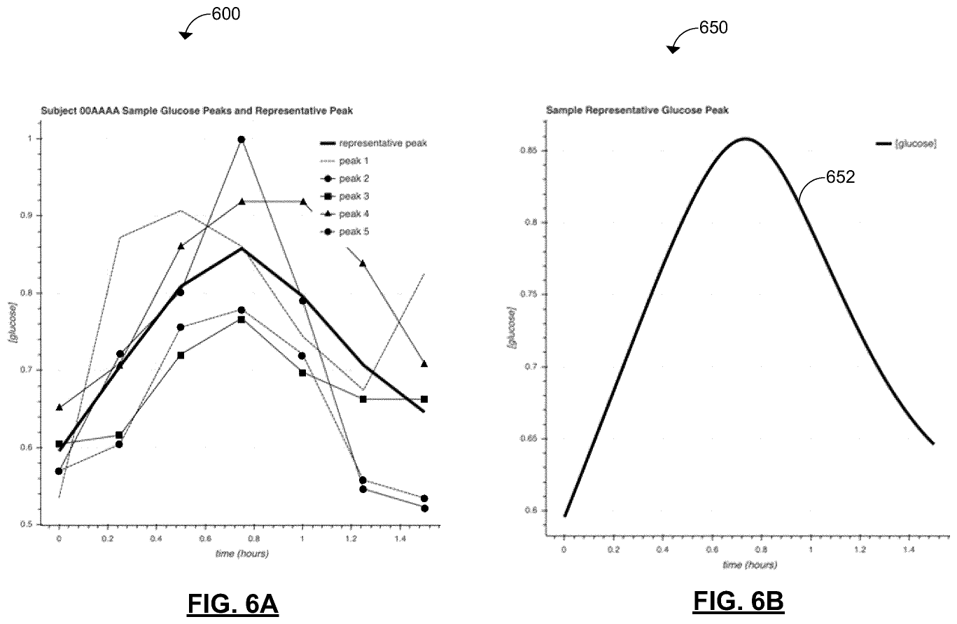

Reference is next made to FIG. 5, there is shown another example diagram 500 of glucose measurement data. The glucose measurement data shown in example diagram 500 may be collected using a glucose measurement device (see 108 in FIG. 1). In the example shown in FIG. 5, time series data for three days (Day 1, Day 2, and Day 3) has been overlaid. The example diagram 500 further includes minimum safe values 504 and maximum safe value 502. The example diagram 500 further includes an average value of the three days (Day 1, Day 2, and Day 3).

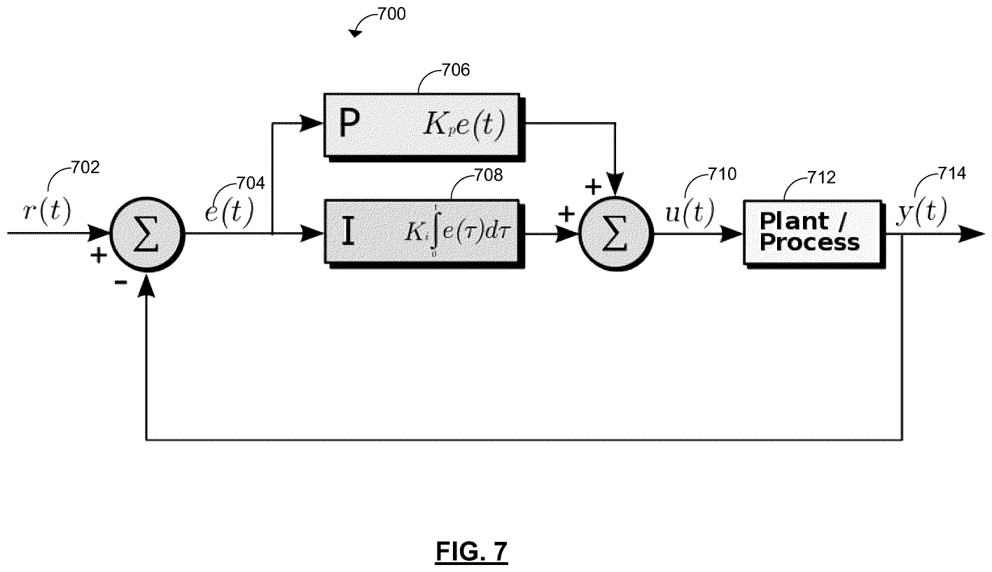

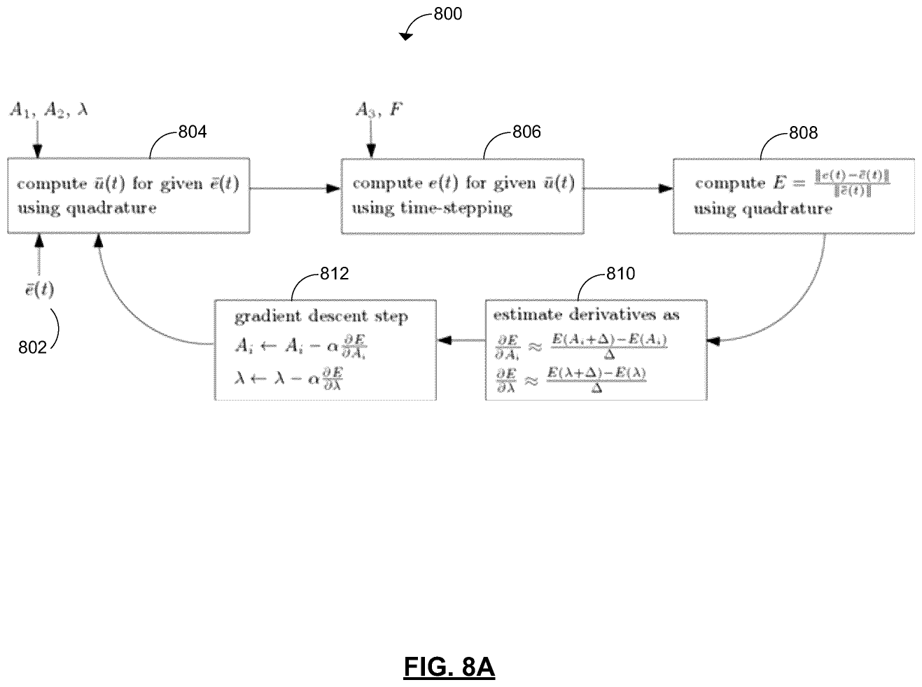

Reference is next made to FIG. 6A, there is shown an example diagram 600 of glucose time series data having overlaid sample peaks. The analysis of the glucose measurements from a user to determine a model may involve selecting one or more curve intervals that correspond to one or more local maxima of the glucose measurements. The one or more curve intervals may be normalized. The one or more curve intervals may be taken from glucose measurements of a single day, or multiple days.

Reference is next made to FIG. 6B, there is shown a representative peak diagram 650 of the glucose time series data in FIG. 6A. The representative peak 652 may be determined based on the normalized one or more curve intervals. The normalized one or more curve intervals may be averaged to determine the representative peak 652.

In one embodiment, a representative curve may be determined based on at least two curve intervals determined from the glucose measurement data. The at least two curve intervals may each have at least three glucose measurements. The at least two curve intervals of the glucose measurement data may be averaged and/or normalized. The averaging may occur before the normalization, or after. The averaging and the normalization may be performed across the glucose measurement data prior to the selection of the at least two curve intervals.

In one embodiment, a representative curve may be determined based on at least 5, 10, 15, 20 or 25 curve intervals, wherein each curve interval comprises at least three glucose measurements. In one embodiment, a representative curve may be determined based on at least 5, 10, 15, 20 or 25 curve intervals, wherein each curve interval comprises at least four glucose measurements. In one embodiment, a representative curve may be determined based on at least 5, 10, 15, 20 or 25 curve intervals, wherein each curve interval comprises at least five glucose measurements.

In one embodiment, frequency of glucose measurements in each curve interval used for determining the representative curve is at least every 20 minutes, every 15 minutes, every 10 minutes or every 5 minutes. In one embodiment, each of the one or more curve intervals may be based on 4, 5, 6 or more than 6 glucose measurements. In one embodiment, the representative curve may be determined based on 3, 4, 5, 6 or more than 6 curve intervals.

The representative peak diagram 650 has a vertical axis of glucose concentration, and a horizontal axis of time units, based on a 15-minute capture interval, or at another capture frequency as disclosed herein.

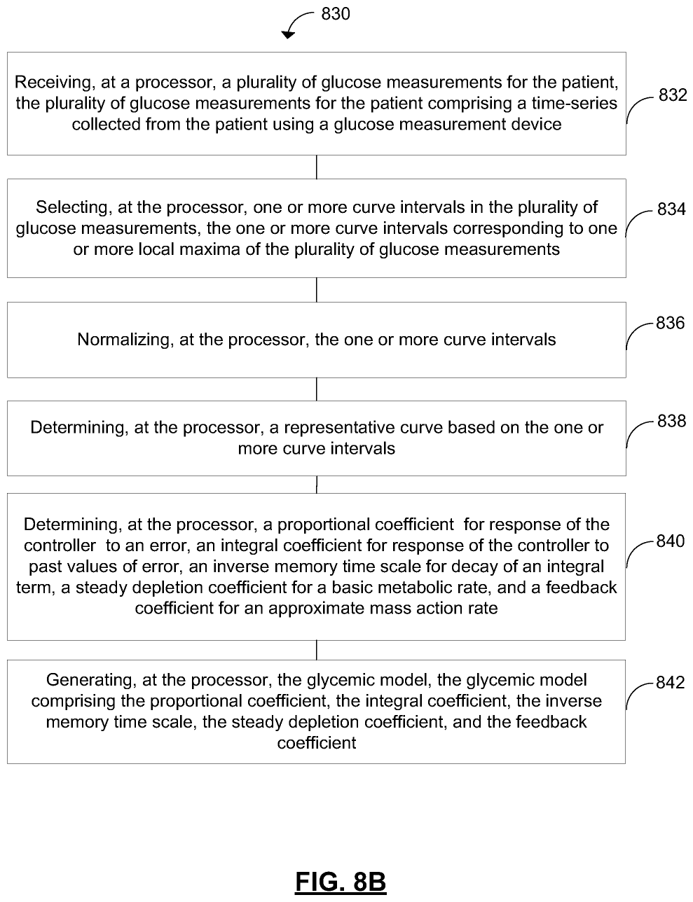

Reference is next made to FIG. 7, showing a proportional-integral (PI) model diagram 700. A PI model is a control loop model that uses feedback, without the derivative term used in the related proportional-integral-derivative (PID) model. The PI has two main constituents, a proportional term and an integral term.

The PI model 700 may have a desired set point r(t) 702 that is the desired or target value for a variable, or process value of a system. Departure of such a variable from its set point may be a basis for error-controlled regulation using negative feedback for control. The set point may be described herein as SP.

A measured process value y(t) 714 may be measured from the system controlled using the PI model. The measured process value may be described herein as PV.

The PI model 700 may determine an error value e(t) 704 that is the difference between the desired set point and the measured process value. The error value may be determined based on the equation e(t)=y(t)−r(t).

The PI model 700l may have a proportional term P 706, represented by Kₚe(t). The proportion term P 706 is proportional to the current value of the error e(t). The proportional term P 706 may have a coefficient Kₚ.

The PI model 700 may have an integral term I 708, represented by Kᵢ∫₀^{t}e(t)dr. The integral term I 708 accounts for past values of the error e(t) 704. The integral term I 708 may have a coefficient Kᵢ.

The PI model 700 may determine a controller value u(t) 710 that may be used as an input to a process 712 in order to provide a correction to adjust the measure process value 714. The controller value u(t) 710 may be continuously updated to provide modulated control for the process 712. The controller value u(t) 710 may be determined based on the proportional term and the integral term. The controller value u(t) 710 may be determined using the equation (t)=Kₚe(t) +Kᵢ∫₀^{t}e(τ)dτ.

The process 712 may be any process involving a feedback loop, including an industrial process or a biological process.

In a preferred embodiment, the PI model is extended to determine a model for glucose homeostasis. The extended PI model comprises two equations, a first equation for the PI model for glucose homeostasis, and a second equation describing a glucose response.

The first equation for modelling glucose homeostasis is given as Equation 1.

$$u ( t ) = {A}_{1} e ( t ) + {A}_{2} {\int }_{- ∞}^{∞} w ( t - {t}^{′} , λ ) e ( {t}^{′} ) {dt}^{′} \qquad ( Equation 1 )$$

The second equation for describing a glucose response is given as Equation 2.

$$\frac{d e}{d t} = - {A}_{3} + F ( t ) - {A}_{4} u ( e + {e}_{sp} ) \qquad ( Equation 2 )$$

As shown in Equation 1 and Equation 2, u(t) is a control value, eₛₚ is the set point blood sugar level, i.e. the level that the feedback system tries to maintain and e is the deviation therefrom. The λ factor is defined as w such that ∫w(τ)dτ=1. The λ factor may be a tunable parameter of the glucose homeostasis model as described herein.

The weight function w may be added to the integral term of Equation 1 that models the influence of past blood sugar levels on the current level of control. The weight function w may be described using exponential decay, namely as described in Equation 3.

$$w ( τ , λ ) = \{ \begin{matrix}0 if τ < 0 \\ {λexp}^{- λ τ} if τ > 0\end{matrix} \qquad ( Equation 3 )$$

The control variable u(t) 710 may respond to the deviation from the set point blood sugar level eₛₚ in proportion to proportional coefficient A₁, and based on its history, with integral coefficient A₂. The influence of past blood sugar levels may decrease exponentially at a rate λ, and λ may be referred to herein as the inverse memory time scale for decay of the integral term. A₁ may be referred to herein as the proportional coefficient. A₂ may be referred to herein as the integral coefficient.

The rate of change of the blood sugar deviation de/dt may be set by three terms, A₃, F(t), and A₄. Firstly, there is a steady depletion modelling the basic metabolic rate, A₃. A₃ may be referred to herein as the steady depletion coefficient. Secondly, F (t) may model food intake and circadian rhythm. F(t) may be referred to as the input function, and may have a Gaussian shape. Finally, there may be feedback from the control mechanism A₄. A₄ may be referred to herein as the feedback coefficient. The feedback may be modelled based on mass action kinetics. In this approach, insulin and blood sugar may act like reactants in a generally uniformly mixed reaction vessel. The rate at which blood sugar is taken out of the system may be proportional to the insulin and total blood sugar concentrations, with an amplitude A₄.

In one embodiment, a general feedback function may be considered, and a Taylor expansion may be performed, retaining only the lowest order terms that depend on the controller.

In Table 1 below, the model parameters are summarized. Two non-dimensional parameters, B₁ and B₂, may characterize the control system and are defined as B₁=A₁/A₂ and B₂=λ/A₄. B₁ may measure the relative influence of the proportional and integral terms of the controller, and B₂ may measure the ratio of time scales that may characterize the decaying influence of past blood sugar levels and the efficiency of the feedback loop. B₁ may be referred to herein as a glucose homeostasis metric. B₂ may be referred to herein as a feedback loop metric.

TABLE 1
Parameters of the glucose homeostasis model with their
meaning and typical range across test subjects.

| Parameter | Meaning | Units |
|---|---|---|
| A₁ | Proportional control term | litre/mmol |
| A₂ | Integral control term | litre/mmol |
| A₃ | Basic metabolic rate | mmol/(litre × Δt) |
| A₄ | Feedback amplitude | 1/Δt |
| λ | Decay rate of the Integral term | 1/Δt |

A constant input F may provide qualitative insight into the behavior of the glucose homeostasis model. In this case, there may be a critical value F* of the input given by Equation 4:

$${F}^{*} = {A}_{3} - \frac{1}{4} {A}_{4} ( {A}_{1} + {A}_{2} ) {e}_{sp}^{2} \qquad ( Equation 4 )$$

The critical value F* may be a peak value. If F<F*, the blood sugar level may decrease monotonically and the homeostasis may fail. In contrast, if F>F*, the success of the homeostatic control may depend on the initial blood sugar level. If it is below e₋^{bar} , the control may also fail. If not, the blood sugar level may approach the stable equilibrium value e₊^{bar}. Here the e_{±}^{bar} critical values are given by Equation 5:

$${e}_{+ / -}^{bar} = - \frac{1}{2} {e}_{sp} + \frac{1}{2} \sqrt{{e}_{sp}^{2} + \frac{4 ( F - {A}_{3} )}{{A}_{4} ( {A}_{1} + {A}_{2} )}} \qquad ( Equation 5 )$$

These e_{±}^{bar} critical values may demonstrate that the modelled homeostasis is stable only if there is sufficient sugar input and if the system does not become overly hypoglycemic.

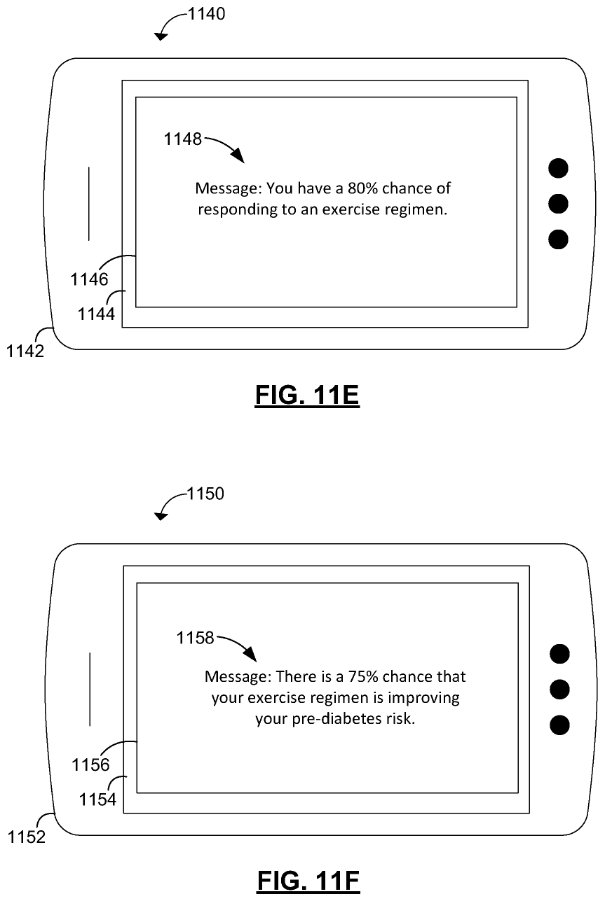

Reference is next made to FIG. 12, which shows a distribution diagram 1200 of the indicator R also referred to a glucose homeostasis metric R. In an alternate embodiment, an indicator R may be determined as given by Equation 10, where σₑ is the standard deviation of all glucose measurements for a given subject and uₘ is the maximum attained by the control variable in the optimal fit.

$$R = \frac{{σ}_{e} ( {A}_{2} - {A}_{1} )}{{u}_{m}} \qquad ( Equation 10 )$$

The indicator, R may indicate the responsiveness of the glycemic control systems. The distribution 1200 shows the R value of subjects, with the values displayed as dots on the horizontal axis, and the distribution displayed as a histogram.

The determined R values appear to have a clear modal value of around R=0, and a positive skew towards higher values. The R indicator may be used as an actionable diagnostic tool, extracted from quasi-continuous glucose measurements in real-time. As shown in FIG. 12, two outliers exist at the high end of the R scale. For these outliers, the proportional and integral terms of the control strategy may work against each other. This may be indicative of a pathological state such as prediabetes.

Furthermore, as set out in Example 3, a higher value of the glucose homeostasis metric R was observed in a subject with Type II diabetes relative to a number of control subjects without known glycemic dysfunction.

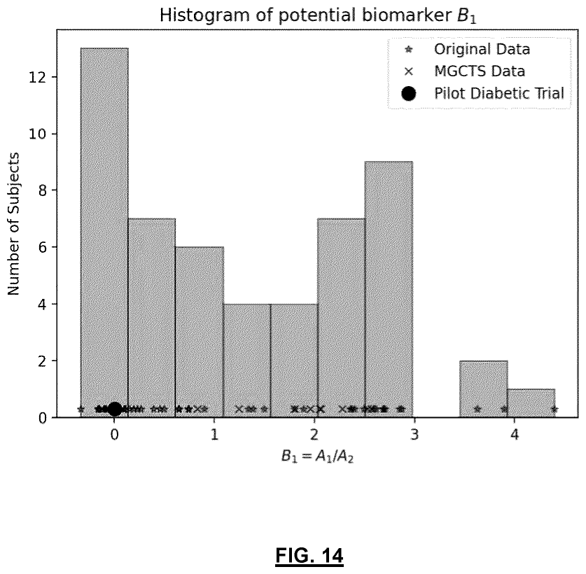

As shown in FIG. 15, the use of the glucose homeostasis metric R was able to distinguish between individuals without any diagnosed glycemic dysfunction and a subject with confirmed Type II diabetes. High values of R may therefore be indicative of diabetes or a pathological state such as prediabetes relative to control values of R from subjects representative of a normal population without glycemic dysfunction.

Reference is next made to FIG. 8A, there is shown an example method diagram 800 for determining a glucose control model. e^{bar}(t) is the error value derived from the representative peak determined for a user.

At 802, an e^{bar}(t) is provided in the form of the representative curve.

At 804, u^{bar}(t) is determined, given e^{bar} (t) and initial approximations for A₁, A₂, and λ using a numerical quadrature (for example, the Midpoint Rule) of the integral from time 0 to the current time, for all available glucose measurements

At 806, given the approximate values for A₁, A₂, and λ, u^{bar}(t), and approximate values for A₃, A₄, and F, e(t) may be determined by time stepping (for example, Euler's method) for the given u^{bar}(t).

At 808, determining an error E, by evaluating E=||e(t)−e^{bar}(t) ||/||e^{bar}(t)|| using quadrature (for example, the Midpoint Rule). E may be a determination of the sum-squared error (SSE) between the vector representation of e^{bar}(t) and a vector representation of e(t).

Based on the representative peak data e^{bar}(t) and the values of A₁, A₂, and λ, u^{bar}(t) may be computed from Equation 1, and this may represent the time course of the control variable corresponding to the representative peak. Using this u^{bar}(t) and the values for A₃, and A₄, as well as a putative Gaussian peak and F(t), e(t) may be determined from Equation 2. This may correspond to the model output generated by the input function F(t) and the control time course u_{bar}(t). If this e(t) coincides with e_{bar}(t), the model parameter values may be said to be generally exact. The error E is the difference between e(t) and e_{bar}(t). Since e(t) and e_{bar}(t) are time series functions (for example, 5 values at 15 min intervals), they may be considered vectors and a vector norm may be used to compute E.

At 810, derivatives may be determined by estimating ∂E/∂Aᵢ and ∂E/∂λ for A₁, A₂, A₃, A₄, and λ according to Equation 6 and Equation 7 respectively. The derivatives may be determined using finite difference approximation. For each derivative, E may be computed twice for slightly difference values of the parameter in question. In one embodiment, the derivative of E with respect to variations in the input function F may be estimated in the same way.

$$\frac{\partial E}{\partial {A}_{i}} \approx \frac{E ( {A}_{i} + Δ ) - E ( {A}_{i} )}{Δ} \qquad ( Equation 6 )$$

$$\frac{\partial E}{\partial λ} \approx \frac{E ( λ + Δ ) - E ( λ )}{Δ} \qquad ( Equation 7 )$$

At 812, a gradient descent may be performed to determine new approximations for A₁, A₂, A₃, A₄, F, and λ, according to equations 8 and 9.

$${A}_{i} ← {A}_{i} - α \frac{\partial E}{\partial {A}_{i}} \qquad ( Equation 8 )$$

$$λ ← λ - α \frac{\partial E}{\partial λ} \qquad ( Equation 9 )$$

The method 800 may be performed iteratively for numerous iterations to determine better approximations for values of A₁, A₂, A₃, A₄, F, and λ. The method 800 may be iteratively performed using gradient descent to determine better approximations for values of A₁, A₂, A₃, A₄, F and λ.

Reference is next made to FIG. 8B, there is shown another example method diagram 830 for determining a glucose control model.

At 832, receiving, at a processor, a plurality of glucose measurements for the patient, the plurality of glucose measurements for the patient comprising a time-series collected from the patient using a glucose measurement device.

At 834, selecting, at the processor, one or more curve intervals in the plurality of glucose measurements, the one or more curve intervals corresponding to one or more local maxima of the plurality of glucose measurements.

At 836, normalizing, at the processor, the one or more curve intervals.

At 838, determining, at the processor, a representative curve based on the one or more curve intervals.

In at least one embodiment, the determining, at the processor, the representative curve may further comprise averaging, at the processor, the one or more normalized curve intervals.

At 840, determining, at the processor, a proportional coefficient A₁ for response of the controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate.

In at least one embodiment, the determining, at the processor, the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate may further comprise determining, at the processor, a first approximate proportional coefficient, a first approximate integral coefficient and a first approximate inverse memory time scale of the representative curve based on an approximation of an integral of the representative curve; determining, at the processor, a first approximate steady depletion coefficient and a first approximate feedback coefficient based on a differential equation of the representative curve, the first approximate proportional coefficient, the first approximate integral coefficient, and the first approximate inverse memory time scale; and determining, at the processor, a first vector comprising the first approximate proportional coefficient, the first approximate integral coefficient, the first approximate inverse memory time scale, the first approximate steady depletion coefficient and the first approximate feedback coefficient.

In at least one embodiment, the determining, at the processor, the proportional coefficient A₁ for response of the controller u(t) to the error e(t), the integral coefficient A₂ for response of the controller u(t) to the past values of error e(t), the inverse memory time scale λ for decay of the integral term, the steady depletion coefficient A₃ for the basic metabolic rate, and the feedback coefficient A₄ for the approximate mass action rate may further comprise determining, at the processor, a second approximate proportional coefficient, a second approximate integral coefficient and a second approximate inverse memory time scale of the representative curve based on the approximation of an integral of the representative curve; determining, at the processor, a second approximate steady depletion coefficient and a second approximate feedback coefficient based on a differential equation of the representative curve, the second approximate proportional coefficient, the second approximate integral coefficient, and the second approximate inverse memory time scale; determining, at the processor, a second vector based on the second approximate proportional coefficient, the second approximate integral coefficient, the second approximate inverse memory time scale, the second approximate steady depletion coefficient and the second approximate feedback coefficient; comparing, at the processor, an error between the first vector and the second vector; and performing, at the processor, a gradient descent to modify the first approximate proportional coefficient, the first approximate integral coefficient, the first approximate inverse memory time scale, the first approximate steady depletion coefficient and the first approximate feedback coefficient.

In one or more embodiments, the determining, at the processor, the first approximate proportional coefficient, the first approximate integral coefficient and the first approximate inverse memory time scale of the representative curve may be based on a midpoint rule approximation of the integral of the representative curve.

In one or more embodiments, the determining, at the processor, the first approximate steady depletion coefficient and the first approximate feedback coefficient may be determined by applying Euler's method to the differential equation of the representative curve, the first approximate proportional coefficient, the first approximate integral coefficient, and the first approximate inverse memory time scale.

At 842, generating, at the processor, the glucose homeostasis model,

the glucose homeostasis model comprising the proportional coefficient A₁, the integral coefficient A₂, the inverse memory time scale λ, the steady depletion coefficient A₃, and the feedback coefficient A₄.

In one or more embodiments, a glucose homeostasis metric may be determined. Various measures of glycemic function may be determined based on one or more coefficients A₁, A₂, A₃, A₄and λ. Optionally, in some embodiments, the measure of glycemic function may also be based on the statistical measure of blood glucose levels for a subjects, such as a standard deviation. For example, in these one or more embodiments, the method may further comprise determining, at the processor, a glucose homeostasis metric B₁, the glucose homeostasis metric B₁ based on the proportional coefficient A₁ and the integral coefficient A₂; and wherein the glucose homeostasis model further comprises the glucose homeostasis metric B₁.

In another embodiment, the method may further comprise determining, at the processor, a glucose homeostasis metric R, the glucose homeostasis metric R based on the proportional coefficient A₁, the integral coefficient A₂, the standard deviation of glucose measurements for a given subject σₑ, and the maximum attained by the control variable in the optimal fit uₘ wherein the glucose homeostasis model further comprises the glucose homeostasis metric R.

The glucose homeostasis metric B₁ may be determined as the product of the proportional coefficient A₁ divided by the integral coefficient A₂.

In one or more embodiments, a feedback loop metric may be determined. In these one or more embodiments, the method may further comprise determining, at the processor, a feedback loop metric B₂, the feedback loop metric B₂ based on the inverse memory time scale term λ and the feedback coefficient A₄; and wherein the glucose homeostasis model further comprises the feedback loop metric B₂.

The feedback loop metric B₂ may be determined by dividing the inverse memory time scale term λ by the feedback coefficient A₄.

In one or more embodiments, the glucose homeostasis metric B₁ and/or the feedback loop metric B₂ may be displayed to a user on a display (see e.g. 204 in FIG. 2).

In one or more embodiments, the glucose homeostasis metric B₁ and/or the feedback loop metric B₂ may be transmitted at a network device (see e.g. 215 in FIG. 2) to a remote service (see e.g. 112 in FIG. 1).

Reference is next made to FIG. 8C, there is shown an example method diagram 860 for using a glucose control model.

At 862, receiving, at a processor, a glucose homeostasis model, the glucose homeostasis model comprising a proportional coefficient A₁, an integral coefficient A₂, an inverse memory time scale λ, a steady depletion coefficient A₃, and a feedback coefficient A₄.

At 864, receiving, at a processor, one or more current glucose measurements.

At 866, determining, at the processor, a glucose message based on the glucose homeostasis model, and the one or more current glucose measurements.

In one or more embodiments, the determining, at the processor, the glucose message based on the glucose homeostasis model, and the one or more current glucose measurements may further comprise determining, at the processor, a glucose screening message, the glucose screening message for predicting a likelihood that a user has a health condition; wherein the glucose message may be the glucose screening message.

In one or more embodiments, the determining, at the processor, the glucose message based on the glucose homeostasis model, and the one or more current glucose measurements may further comprise determining, at the processor, a glucose diagnostic message, the glucose diagnostic message for a glucose diagnostic measurement; wherein the glucose message may be the glucose diagnostic message.

In one or more embodiments, the determining, at the processor, the glucose message based on the glucose homeostasis model, and the one or more current glucose measurements may further comprise determining, at the processor, a glucose predictive message, the glucose predictive message for predicting that a user will develop a health condition; wherein the glucose message may be the glucose predictive message.

In one or more embodiments, the determining, at the processor, the glucose message based on the glucose homeostasis model, and the one or more current glucose measurements may further comprise determining, at the processor, a glucose prognostic message, the glucose prognostic message for predicting whether a health condition of a user is more likely to respond to an intervention; wherein the glucose message may be the glucose prognostic message.

In one or more embodiments, the determining, at the processor, the glucose message based on the glucose homeostasis model, and the one or more current glucose measurements may further comprise determining, at the processor, a glucose response message, the glucose response message for predicting a performance of a current intervention; wherein the glucose message may be the glucose response message.

At 868, displaying, at a display device, the glucose message.

Reference is next made to FIG. 11A, there is shown an example of a user interface drawing 1100. The user interface 1106 is shown on the display 1104 of mobile device 1102. The user interface 1106 may include a generated B₁ metric 1103, that may be visualized to a user using a variety of user interface methods such as slider graph 1105. The user interface 1106 may include a generated B₂ metric 1109, that may be visualized to a user using a variety of user interface methods such as slider graph 1107.

Reference is next made to FIG. 11B, there is shown another example of a user interface drawing 1110. The user interface 1116 is shown on the display 1114 of mobile device 1112. The user interface 1116 may display a glucose screening message 1118 to a user. The glucose screening message 1118 may be for predicting a likelihood that a user has a health condition, for example “Message: You have a 38% likelihood of having type 2 diabetes”.

Reference is next made to FIG. 11C, there is shown another example of a user interface drawing 1120. The user interface 1126 is shown on the display 1124 of mobile device 1122. The user interface 1126 may display a glucose diagnostic message 1128 to a user. The glucose diagnostic message 1128 may be for a glucose diagnostic measurement, for example “Message: The patient has a 38% chance of having type 2 diabetes”.

Reference is next made to FIG. 11D, there is shown another example of a user interface drawing 1130. The user interface 1136 is shown on the display 1134 of mobile device 1132. The user interface 1136 may display a glucose predictive message 1138 to a user. The glucose predictive message 1138 may be for predicting that a user will develop a health condition, for example “Message: You have a 24% chance of developing type 2 diabetes in the next 2 years”.

Reference is next made to FIG. 11E, there is shown another example of a user interface drawing 1140. The user interface 1146 is shown on the display 1144 of mobile device 1142. The user interface 1146 may display a glucose prognostic message 1148 to a user. The glucose prognostic message 1148 may be for predicting whether a health condition of a user is more likely to respond to an intervention, for example “Message: You have an 80% chance of responding to an exercise regimen”.

Reference is next made to FIG. 11F, there is shown another example of a user interface drawing 1150. The user interface 1156 is shown on the display 1154 of mobile device 1152. The user interface 1156 may display a glucose response message 1158 to a user. The glucose response message 1158 may be for predicting a performance of a current intervention, for example “Message: There is a 75% chance that your exercise regimen is improving your pre-diabetes risk”.

### EXAMPLES

### Example 1: Use of a Continuous Glucose Monitor for Modeling Glucose Homeostasis as a Control System in Non-Diabetic Adults

### Participants

A total of 31 participants completed the study (13 females; 18 males; age range =19-50 years, M (age)=32.3, SD (age)=7.3). Participant race included 19 (61.3%) Caucasian, 10 (32.3%) Asian, 1 (3.2%) Hispanic, and 1 (3.2%) mixed race (Caucasian and African American). All participants were employees of Klick Inc. (Toronto, Canada) and were recruited via the company's online intranet system. The study received full ethics approval from an independent ethics committee and all participants signed informed consent.

Exclusion criteria were participants below the age of 18, those who were diagnosed with any mental or physical medical condition of any kind (chronic or acute), those taking any form of prescription medication, and those who were pregnant or breastfeeding. This sample of participants had an average body mass index of 25.8 (SD=6.1), an average resting blood pressure of 120/75 mm Hg, and an average resting heart rate of 70 bpm. Table 2 provides a summary of the physiological data collected for each subject who participated in the study.

TABLE 2
Summary and physiological data for the 31 study participants.

| | | | | Systolic | Diastolic | Resting |
|---|---|---|---|---|---|---|
| | # Days | | | Blood | Blood | Heart |
| | Sensor | | | Pressure | Pressure | Rate |
| | Lasted | Age | BMI | (mmHg) | (mmHg) | (bpm) |
| Average | 12.7 | 32.3 | 25.8 | 120.1 | 75.4 | 70.3 |
| High/Low | 14/7 | 50/19 | 42.4/17.9 | 163/94 | 98/59 | 93/52 |

### Apparatus

The FreeStyle Libre™ flash glucose monitoring system (available from Abbott Diabetes Care) was used to measure real-time, continuous interstitial glucose levels with a minimally invasive 5 mm flexible filament inserted into the posterior upper arm. The sensor works based on the glucose-oxidase process by measuring an electrical current proportional to the concentration of glucose. The device contains a sensor which is attached to the posterior region of the upper arm with an adhesive patch, and a handheld reader device which downloads data from the sensor via near-field communication. Interstitial glucose concentrations (in mmol/L) are captured by the sensor every 15 min and/or when users scan the sensor using the handheld device. The handheld device requires users to scan the sensor at least every 8 hours, otherwise previous data are overwritten by the sensor. The system has a lifespan that restricts sensor wear to 14 consecutive days, after which the handheld device will no longer download data from the sensor.

### Data Collection

At the beginning of the 14-day study period, participants completed self-report health questionnaires and demographic information, and had some physiological variables measured, including height, weight, body mass index (BMI), body fat %, resting blood pressure, and resting heart rate.

Participants were then outfitted with the FreeStyle Libre™ flash glucose monitor, and instructed on its use. Participants were instructed to scan the sensor with the handheld device at least once every 8 hours to minimize data loss. Missing data were anticipated as participants may have slept over 8 hours, so they were encouraged to scan the device before going to sleep and immediately upon waking.

### Model of Glycemic Control

The model comprises two equations, one for the PI controller and one describing the response of the blood sugar level. They are given by

$$u ( t ) = {A}_{1} e ( t ) + {A}_{2} {\int }_{- ∞}^{∞} w ( t - {t}^{′} , λ ) e ( {t}^{′} ) {dt}^{′} \qquad ( Equation 1 )$$

$$\frac{d e}{d t} = - {A}_{3} + F ( t ) - {A}_{4} u ( e + {e}_{sp} ) . \qquad ( Equation 2 )$$

In Equation 1 and Equation 2, , u(t) is a control value, eₛₚ is the set point blood sugar level, i.e. the level that the feedback system tries to maintain and e is the deviation therefrom. The A factor is defined as w such that that ∫w(τ)dt=1. In one embodiment, the λ factor is a tunable parameter of the glucose homeostasis model as described herein.

The weight function w models the influence of past blood sugar levels on the current level of control. It is given by an exponential decay, namely Equation 3:

$$w ( τ , λ ) = \{ \begin{matrix}0 if τ < 0 \\ {λexp}^{- λ τ} if τ > 0\end{matrix} \qquad ( Equation 3 )$$

The control variable responds to the deviation from the set point blood sugar level in proportion, with amplitude A₁, and based on its history, with amplitude A₂. The influence of past blood sugar levels wanes exponentially at a rate λ. The rate of change of the blood sugar deviation is set by three terms. Firstly, there is a steady depletion modelling the basic metabolic rate, A₃. Secondly, F(t) models food intake and the circadian rhythm. Finally, there is the feedback from the control mechanism. This has been modelled based on mass action kinetics. In this simple approach, insulin and blood sugar are imagined to act like reactants in a perfectly mixed reaction vessel. The rate at which blood sugar is taken out of the system is then proportional to the insulin and total blood sugar concentrations, with an amplitude A₄. An alternative motivation for this form of the feedback is to take into consideration that fact that our model should hold for small to moderate deviation from the set point blood sugar level.

In one embodiment, this may be considered a general feedback function and a Taylor expansion performed, retaining only the lowest order terms that depend on the controller.

Table 3 provides a summary of the parameters of the model. Two non-dimensional parameters that characterize the control system are B₁=A₁/A₂ and B₂=λ/A₄. They measure the relative influence of the proportional and integral terms of the controller and the ratio of time scales that characterize the decaying influence of past blood sugar levels and the efficiency of the feedback loop.

TABLE 3
Parameters of the glucose homeostasis model with their
meaning. Here, Δt = 15 minutes which was the interval
between two measurements of the glucose monitoring device.

| Parameter | Meaning | Units |
|---|---|---|
| A₁ | Proportional control term | litre/mmol |
| A₂ | Integral control term | litre/mmol |
| A₃ | Basic metabolic rate | mmol/(litre × Δt) |
| A₄ | Feedback amplitude | 1/Δt |
| λ | Decay rate of the Integral term | 1/Δt |

Some qualitative insight into the behaviour of the model may be obtained by considering a constant input F. In this case, there is a critical value of the input given by:

$${F}^{*} = {A}_{3} - \frac{1}{4} {A}_{4} ( {A}_{1} + {A}_{2} ) {e}_{sp}^{2} \qquad ( Equation 4 )$$

If F<F*, the blood sugar level decreases monotonically and the homeostasis fails. In contrast, if F>F*, the success of the homeostatic control depends on the initial blood sugar level. If it is below e₋^{bar}, the control also fails. If not, the blood sugar level will approach the stable equilibrium value e₊^{bar}. Here the critical values e_{±}^{bar} are given by:

$${e}_{+ / -}^{bar} = - \frac{1}{2} {e}_{sp} + \frac{1}{2} \sqrt{{e}_{sp}^{2} + \frac{4 ( F - {A}_{3} )}{{A}_{4} ( {A}_{1} + {A}_{2} )}} \qquad ( Equation 5 )$$

This demonstrates that the modelled homeostasis is stable only if there is sufficient sugar input and if the system does not become overly hypoglycemic.

### Data Analysis

For each participant, glucose data were recorded for 14 days. Given the 15-minute interval between readings, this accounted for approximately 1000 data points per participant.

From this time series, a number of peaks were manually selected. The representative peak for a given participant was then taken to be the average over the selected peaks. This procedure is demonstrated in FIG. 6. The averaging eliminates much of the noise due to measurement error and provides a sufficiently smooth target for the model fitting.

The procedure used for fitting the model to the representative peak is illustrated in FIG. 8A. The parameters of the model were iteratively updated to minimize the difference between the representative peak and the time series of blood glucose produced by the model. First, the time series of the control variable was computed from the input peak using a simple quadrature rule (right point rule) to evaluate the integral. Once the control variable is known, the equation was time-stepped for the blood glucose with Euler's rule. Any other rule can be used, but Euler's rule with a time-step of 15 minutes, coinciding with the automated measurements, avoids the need for interpolation.

From the time series of blood glucose produced by the model the error of the fit, E, was computed. Simple gradient descent was used to minimize E, approximating the sensitivity of the error function to changes in the parameters by finite differences. This method is particularly simple to implement, but other methods, such as pattern search or quasi-Newton methods can be used equally well.

For the time-stepping the time series of the input function, F(t), was also needed. Since this experiment was not controlled, in the sense that the participants were not required to eat or drink specific amounts or kinds of food at set times, there is no way of estimating the input a priori. The input function was therefore assumed to have a Gaussian shape and its peak value was added to the list of parameters tuned in the gradient descent loop.

The minimization of the error requires tweaking several auxiliary parameters, such as the learning rate of the gradient descent and the finite difference parameter. It was observed that the results are rather insensitive to these details of the numerical algorithm. With a learning rate around 0.001, a relative error of a few percent is reached after about b 10,000 iterations, which only takes the order of seconds on a modest laptop computer.

### Controller Coefficient

From the parameters determined by the fitting procedure, a dimensionless parameter is extracted that reflects the balance between the proportional and integral components of the controller, i.e. are B₁=A₁/A₂. Generally speaking, it is expected that for large values of B₁, the control will act faster but less smoothly than for small values of B₁. Without being limited by theory, it is expected that for a healthy test subject A₁ and A₂ will be positive and both the proportional and integral components of the control act to push the blood glucose level to its target value. A negative value of B₁ indicates that the representative peak has a plateau structure, with a prolonged high of the blood glucose level. This can only occur in the model if the proportional and integral terms approximately cancel each other out, which requires A₁ and A₂ to have an opposite sign.

This controller coefficient may provide a metric for comparing non-diabetic and diabetic subjects and B₁ may also be used for differentiating between subjects and creating inter-subject classes.

### Results

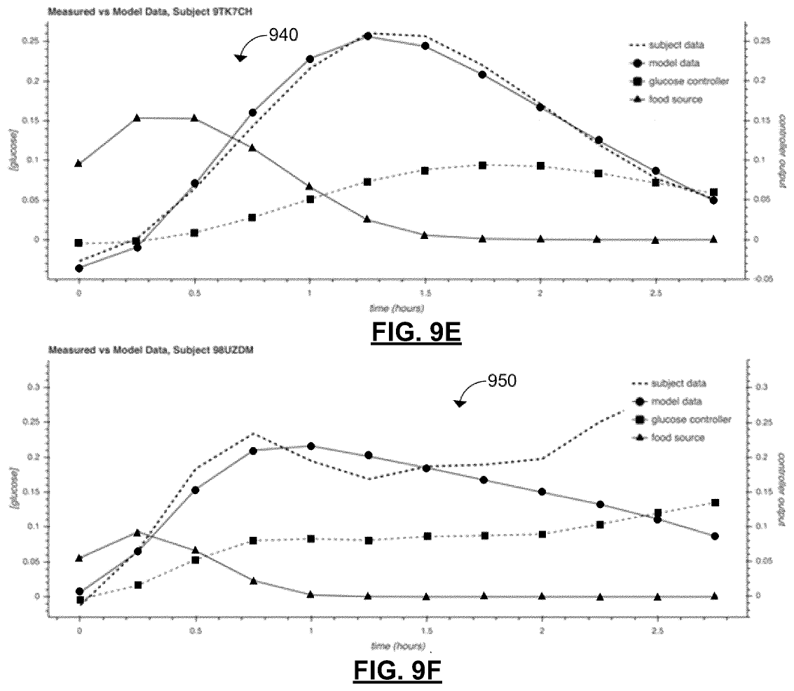

FIG. 9 provides data including a representative curve of measured glucose values and model data for six subjects who participated in the study. For each subject, parameters were tuned such that the minimum error is obtained between ebar and e(t). A value which represents the error between the model and data (E) was calculated—this value is obtained from taking the L2 norm of the difference between the model and data vectors and dividing by the length of the ebar vector. This value shows, for example, that the fit between model and subject data for subject 00AAAA (0.0043) (FIG. 9A) is better than that for subject 8XNLJH (0.0309) (FIG. 9B); upon inspection, it is also clear that the fit for subject 00AAAA is better.

In most plots, it was observed that a close fit is met between subject data and model data. The plot corresponding to Subject 00AAAA (FIG. 9A) appears to be the most optimal fit by inspection; however, this is misleading due to y-axis scaling. From determination of all E-values, subject 8AQUF4 (FIG. 9C) has the best model-to-data fit of all subjects considered, as its E-value is the lowest of those calculated. Conversely, the plot corresponding to 9R39VW (FIG. 9D) appears to be the least-best fit of all subjects modeled, and this is further verified in its E-value of 0.110.

### Separation of Subjects by Class

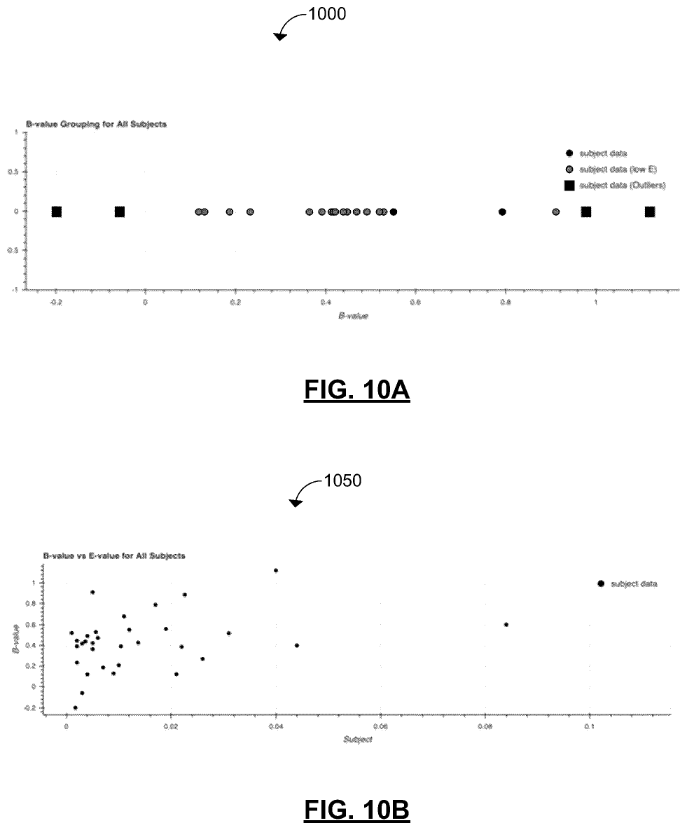

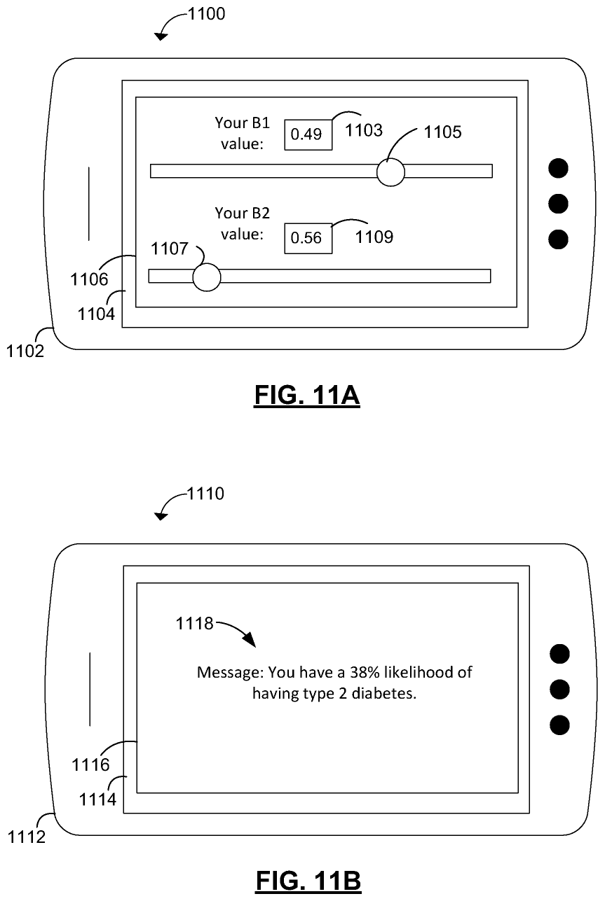

Table 4 provides B₁ and E values for each subject. FIG. 10A provides a plot of B-values for each subject.

FIG. 10A shows that in subject models with E-values cut-off at E=0.01 (i.e. low error between subject data and model data) there is grouping into a normal range and outlier range. If only accurate models are taken into account, the normal range falls in the interval [0.2,0.6]; furthermore, the outlier range falls in the interval [−0.2,0]. It is possible that the outliers have a condition which affects their B-value and therefore their homeostatic controller.

FIG. 10B shows the relationship between the B₁-value and E-value for each subject who participated in the study.

TABLE 4
Values of B₁ and E for each subject who participated in the study.

Subject Name

| | 00AAAA | 8XNLJH | 94TFJR | 8Y99WR | 8YC85H | 8YC830 | 9R39VW |
|---|---|---|---|---|---|---|---|
| B₁-value | 0.49 | 0.234 | 0.391 | 0.55 | 1.12 | 0.79 | 0.98 |
| E-value | 0.004 | 0.002 | 0.002 | 0.012 | 0.04 | 0.017 | 0.11 |

| | Subject Name | | | | | | |
|---|---|---|---|---|---|---|---|
| | 9TK7CH | 9TQA10 | 8AQUF4 | 8CA758 | 8X9MZ4 | 48323W | 8CA77R |
| B₁-value | 0.437 | 0.528 | −0.2 | 0.422 | −0.058 | 0.471 | 0.3907 |
| E-value | 0.0036 | 0.0056 | 0.0017 | 0.005 | 0.003 | 0.006 | 0.0104 |

| | Subject Name | | | | | | |
|---|---|---|---|---|---|---|---|
| | 81XT20 | 94U0MW | 821E1W | 981Z38 | 9831M8 | 98MTZM | 8YC848 |
| B₁-value | 0.515 | 0.364 | 0.426 | 0.445 | 0.886 | 0.398 | 0.519 |
| E-value | 0.031 | 0.005 | 0.0137 | 0.002 | 0.0226 | 0.044 | 0.001 |

| | Subject Name | | | | | | |
|---|---|---|---|---|---|---|---|
| | 8XNLL4 | 98VVGM | 7V9G14 | 8CA8Z4 | 8D1F78 | 82GJLD | 81XT1M |
| B₁-value | 0.679 | 0.558 | 0.599 | 0.187 | 0.27 | 0.123 | 0.418 |
| E-value | 0.011 | 0.019 | 0.084 | 0.007 | 0.026 | 0.021 | 0.003 |

| | Subject Name | | | | | |
|---|---|---|---|---|---|---|
| | 98UZDM | 82GJMW | 9TZ6DR | 9U0Z4R | 9TQA38 | 00BBBB |
| B₁-value | 0.911 | 0.13 | 0.386 | 0.209 | 0.121 | 0.521 |
| E-value | 0.005 | 0.009 | 0.022 | 0.01 | 0.004 | 0.0104 |

### Example 2: Identification of Subjects with Dysfunctional Glucose Homeostasis

Two of the subjects who participated in the study [8AQUF4 and 8X9MZ4] were observed to a have a different B₁ value relative to all the other subjects. The B₁ value is a dimensionless coefficient that devised to assess the effectiveness of the controller. In other words, the B₁ value identifies the effectiveness of the homeostasis function for that individual.

As shown in Table 4 and FIG. 10A, the B₁-values of the subjects who participated in the study were small positive numbers (0.1-0.002), while two outliers had negative B₁ values (−0.2 and −0.058).

Subjects with pre-diabetes may be identified based on a fasting glucose level from 100 to 125 mg/dl (5.6 to 7.0 mmol/L), while a fasting glucose level of 126 mg/dL (7.0 mmol/L) or higher indicates type 2 diabetes. Further criteria for glycemic dysfunction indicative of pre-diabetes or diabetes includes glucose levels following a glucose tolerance test of 140 to 199 mg/dl (7.8 to 11.0 mmol/L) which may be considered prediabetes and a glucose level of 200 mg/dl (11.1 mmol/L) or higher which indicates type 2 diabetes.

While subjects were excluded from participating in the study if they presented with a diagnosis of diabetes, analysis of the raw continuous data for the two subjects with negative B values suggests that they may be at risk of diabetes or pre-diabetes. In particular, visual inspection of the glucose time series data for subjects 8AQF4 and 8X9MZ4 indicated high glucose levels in the early morning which may reflect fasting glucose levels. Furthermore, visual inspection of the glucose time series data glucose data for subjects 8AQF4 and 8X9MZ4 also indicated periodic spikes in glucose levels which may indicate poor performance in a glucose tolerance test and possible pre-diabetes or diabetes.

### Example 3: MGCTS and Pilot Diabetic Trial

A separate cohort of 12 subjects was recruited for a second study (referred to herein as the “MGCTS” study) using a similar apparatus (FreeStyle Libre™ CGM), data collection and model of glycemic control as the “original” study described in Example 1. Physiological and demographic details for subjects in the MGCTS study are presented in Tables 5 and 6. All 12 subjects did not identify as smoking or consuming alcohol. Blood pressure and heart rate were determined for each subject on two separate occasions.

In addition, a single white Caucasian subject previously diagnosed with Type II diabetes was recruited for a pilot diabetic trial using a similar apparatus, data collection and model of glycemic control as described in Example 1. The diabetic subject was male; age 68; White/Caucasian; Height 5′8″ (172 cm); weight 266 lbs (120 kg); and BMI=40.4.

TABLE 5
Summary and physiological data for the cohort of 12 study participants (MGCTS).

| | | | | | | Systolic | | Diastolic | | Resting | |
|---|---|---|---|---|---|---|---|---|---|---|---|
| | | | | | | Blood | | Blood | | Heart | |
| | | | | | | Pressure | | Pressure | | Rate | |
| | Age | | Height | Weight | | (mmHg) | | (mmHg) | | (bpm) | |
| ID | (years) | Sex | (m) | (kg) | BMI | 1st | 2nd | 1st | 2nd | 1 st | 2nd |
| AVD017 | 39 | F | 1.50 | 60 | 26.7 | 110 | 118 | 70 | 70 | 78 | 72 |
| BIM018 | 22 | F | 1.50 | 50 | 22.2 | 110 | 118 | 70 | 78 | 70 | 78 |
| DSM006 | 44 | F | 1.62 | 60 | 22.9 | 118 | n/a | 78 | n/a | 78 | n/a |
| GDP007 | 36 | M | 1.72 | 78 | 26.4 | 113 | 118 | 68 | n/a | 76 | 78 |
| JMB026 | 49 | M | 1.60 | 72 | 28.1 | 120 | 118 | 80 | 78 | 79 | 82 |
| KJB013 | 23 | F | 1.54 | 48 | 20.2 | 114 | 115 | 60 | n/a | 77 | 78 |
| MNB009 | 29 | F | 1.54 | 48 | 20.2 | 115 | 118 | 70 | n/a | 79 | 80 |
| PSG016 | 22 | F | 1.52 | 59 | 25.5 | 118 | 120 | 80 | 80 | 80 | 78 |
| PSK012 | 21 | F | 1.54 | 50 | 21.1 | 119 | 120 | 68 | n/a | 81 | 80 |
| UBR011 | 32 | F | 1.57 | 70 | 28.4 | 122 | n/a | 82 | n/a | 72 | n/a |
| VJB025 | 44 | F | 1.54 | 60 | 25.3 | 118 | 118 | 68 | 70 | 82 | 78 |
| SBG010 | 27 | F | 1.52 | 55 | 23.8 | 118 | n/a | 78 | n/a | 80 | n/a |

TABLE 6
Fasting blood sugar levels, oral glucose tolerance test and
HbA1c levels for the cohort of 12 study participants (MGCTS).

| | Fasting Blood | Oral Glucose | |
|---|---|---|---|
| ID | Sugar Level | Tolerance Test | HbA1c (%) |
| AVD017 | 108.3 | 116.7 | 5.4 |
| BIM018 | 96.6 | 114.4 | 4.5 |
| DSM006 | n/a | n/a | n/a |
| GDP007 | 81.3 | 117.4 | 5.0 |
| JMB026 | 101.4 | 101.7 | 5.3 |
| KJB013 | 74.4 | 80.8 | 5.0 |
| MNB009 | 97.9 | 79.1 | 5.3 |
| PSG016 | 100.1 | 128.6 | 5.2 |
| PSK012 | 77.0 | 85.0 | 5.1 |
| UBR011 | n/a | n/a | n/a |
| VJB025 | 108.6 | 111.6 | 5.1 |
| SBG010 | n/a | n/a | n/a |

### Results

Values for A₁, A₂, B₁ and R as determined for 11 of the 12 participants in the MGCTS study are shown in Table 7. One participant was excluded as the subject dropped out of the study shortly after it began. Values of A₁, A₂, B₁ and R as determined for the diabetic subject are shown in Table 8.

TABLE 7
Determined values of A₁, A₂, R and B1 for each of
the 11 participants who completed the study.
MGCTS

| A₁ | A₂ | R | B₁ |
|---|---|---|---|
| 0.56953 | 0.27605 | −0.34971 | 2.06312 |
| 0.03944 | 0.45261 | 0.70116 | 0.08715 |
| −0.05087 | 0.42738 | 0.89360 | −0.11902 |
| 0.52975 | 0.20934 | −0.27201 | 2.53052 |
| 0.66312 | 0.25813 | −0.33729 | 2.56890 |
| 0.44661 | 0.53802 | 0.09783 | 0.83010 |
| 0.53275 | 0.42733 | −0.07525 | 1.24669 |
| 0.66243 | 0.29092 | −0.43515 | 2.27701 |
| 0.60309 | 0.29347 | −0.46839 | 2.05508 |
| 0.41267 | 0.22931 | −0.43363 | 1.79962 |
| 0.67624 | 0.34498 | −0.31054 | 1.96023 |

TABLE 8
Determined values of A₁, A₂, R and B1 for the diabetic study participant.
Diabetic Trial

| A1 | A2 | R | B1 |
|---|---|---|---|
| 0.00263 | 0.37426 | 1.21567 | 0.00704 |

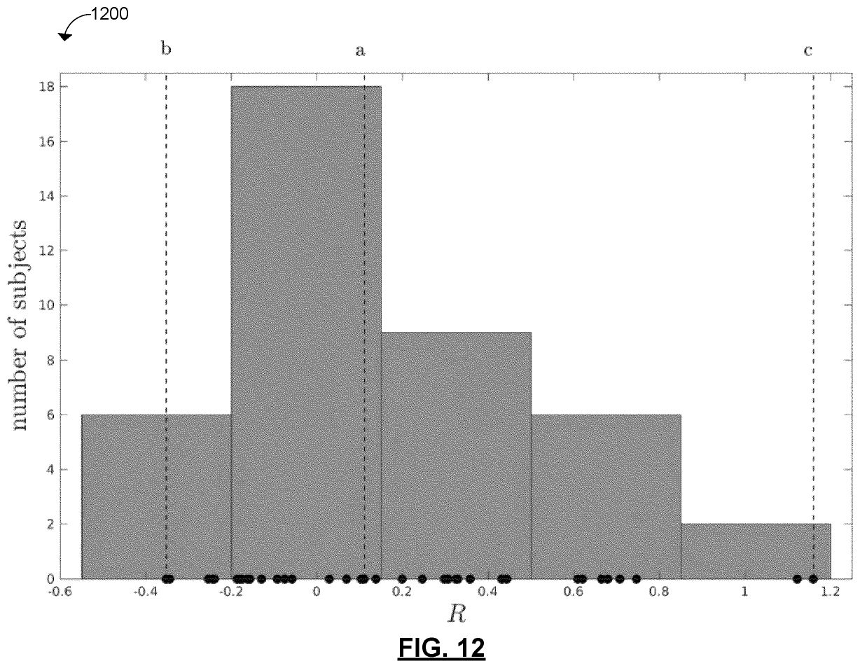

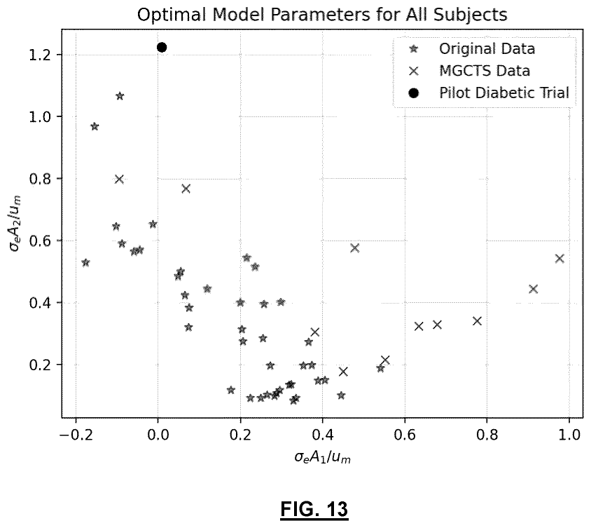

FIG. 13 shows a plot of values for all of the subjects in the original study (Example 1) along with the 11 subjects from the MGCTS study and the diabetic subject. Notably, A₂ appears to be highest in the diabetic subject who also presented with a low value of A₁. FIG. 14 shows histogram of the biomarker B (B=A₁/A₂) with the diabetic subject presenting with a low value of B near 0. Finally, FIG. 15 shows the distribution of biomarker R with the diabetic subject showing the highest value of R.

The use of metrics based on A₁ and A₂ (such as R or B₁) therefore appear to be indicative of glycemic control in human subjects and may be used to identify subjects with glucose homeostasis dysfunction such as diabetes.

### Example 4: Identification of Glucose Homeostasis Phenotypes and Prediabetic Subphenotypes

Blood glucose data was collected for a sample of ˜125 subjects using a continuous glucose monitoring (CGM) device (FreeStyle Libre™ CGM) along with demographic and physiological information. Each subject also underwent an oral glucose tolerance test (OGTT) and was tested for fasting blood glucose and hemoglobin A₁C levels (HbA₁c) levels.

Subjects were identified as normal if they had an Oral Glucose Tolerance Test of less than 140 mmol/ L, a Resting Blood Glucose of less than 100 mmol/L, and an HbA₁c of less than 5.7. Subjects were identified as diabetic if they had a Oral Glucose Tolerance Test of greater than 200 mmol/L, a RBG of greater than 125 mmol/L, and an HbA₁c greater than 6.4. Subjects not identified as normal or diabetic were identified as prediabetic.

Representative curves were generated for each subject and then fitted to the model of glycemic control as described in Example 1 resulting in the determination of values for a proportional coefficient A₁, an integral coefficient A₂, an inverse memory time scale λ, a steady depletion coefficient A₃, and a feedback coefficient A₄.

The two primary parameters of the glucose homeostasis model are denoted by A₁, the coefficient of proportionality, and A₂, the integral coefficient or memory coefficient. A₁ can be interpreted as the coefficient that controls the instantaneous response of blood glucose fluctuations, and A₂ can be interpreted as a feedback mechanism that accounts for recent fluctuations or alternatively the coefficient that controls the inertial response of blood glucose fluctuations. Therefore it is expected that the values of A₁ and A₂ may decrease as an individual becomes more and more diabetic.

Since gender specific hormones play a noticeable role in the regulation of blood glucose levels and estrogen is known to increase insulin sensitivity, subjects were split up into male and female cohorts.

Males identified as pre-diabetic were observed to perform better in OGTT compared to the prediabetic female group despite having the same average HbA₁c values (150.6 vs. 168.6 for OGTT, 105 vs. 106 for resting glucose)

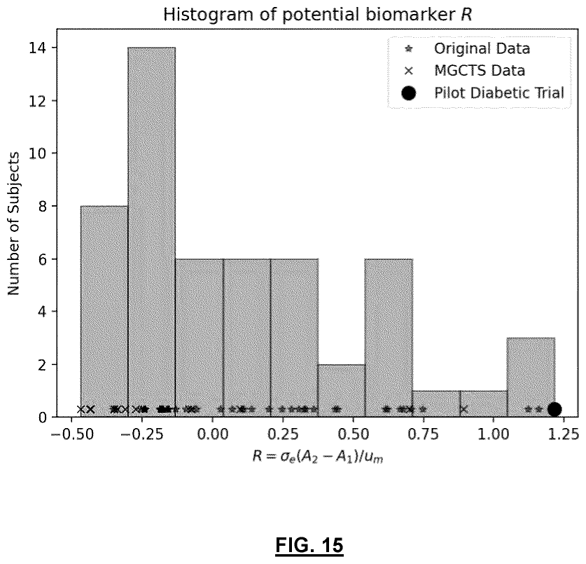

As shown in FIG. 16, A₁ values for subjects identified as prediabetic were observed to cluster in females above and below 0.3, and in males above and below 0.25.

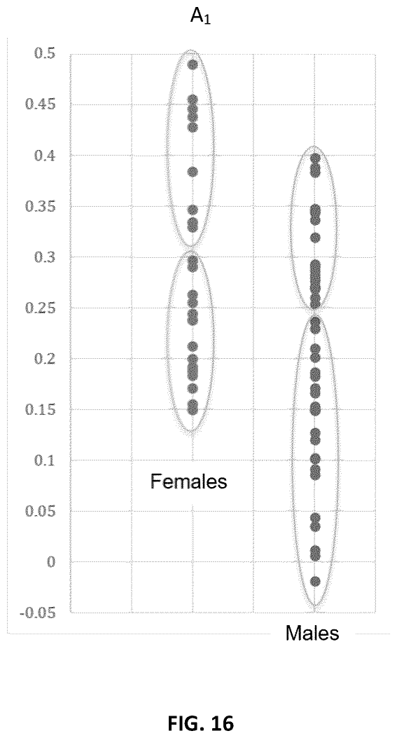

Further investigations were performed to identify additional digital biomarkers suitbale for classifying prediabetic subjects into different phenotypes. As shown in FIG. 17, prediabetic subjects exhibited clustering above and below a value of 0.6 for A₂/λ for both sexes.

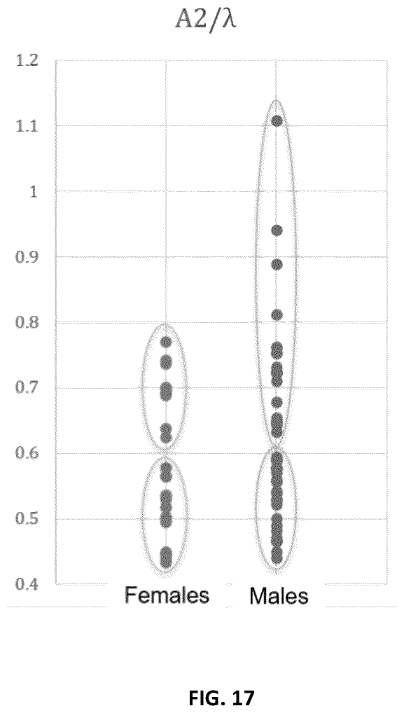

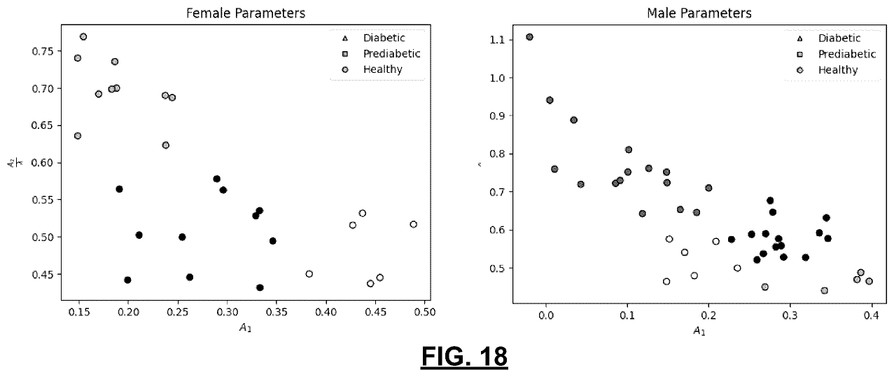

FIG. 18 and FIG. 19 show plots of A₂/λ vs. A₁ for prediabetic subjects (FIG. 18) and for all subjects (FIG. 19).

k-means clustering was also used to separate subjects into clusters and prediabetic subphenotypes based on their proximity to cluster centres. If the cluster center A₁ and A₂/λ are denoted as A₁c and A₂lc, and each individual's parameters points are denoted A₁p and A₂lp, the distance between the point and the center is calculated as follows:

$$d = \sqrt{{( A 1 c - A 1 p )}^{2} + {( A 2 lc - A 2 lp )}^{2}}$$

Female cluster centers were observed at positions corresponding to (A₁, A₂/λ): (0.4393, 0.4827), (0.1901, 0.6970) and (0.2769, 0.5076) and male cluster centers were obtained at positions corresponding to (A₁, A₂/λ): (0.1175, 0.7216), (0.3556, 0.4626), (0.2886, 0.5787) and (0.1830, 0.5217).

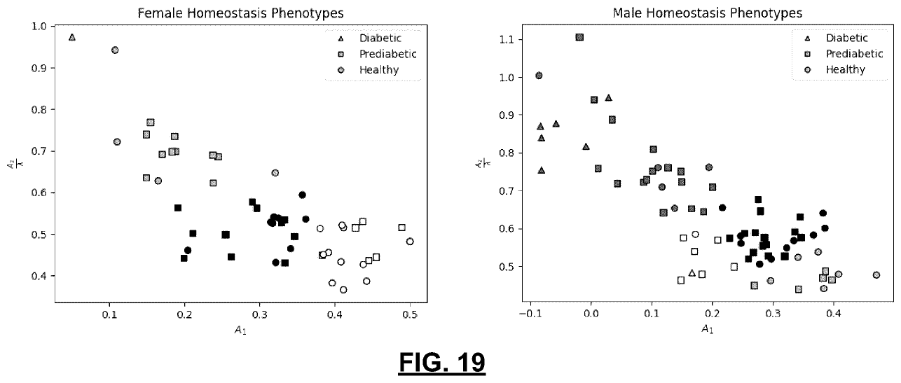

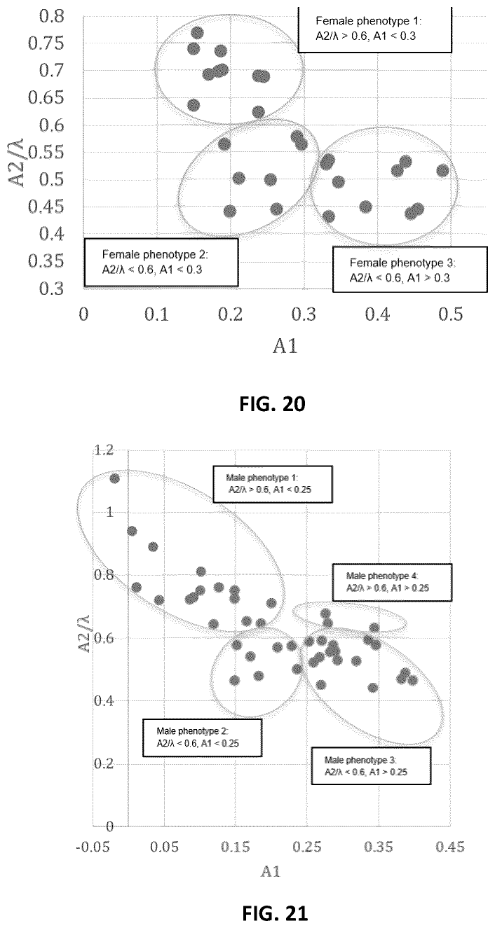

FIG. 20 shows a plot of A₂/λ vs. A₁ for female prediabetic subjects identifying three separate prediabetic subphenotypes. FIG. 21 shows a plot of A₂/λ vs. A₁ for male prediabetic subjects identifying four separate prediabetic subphenotypes.

In terms of the physiological manifestations of these phenotypes, a high value for A₁ represents a strong instantaneous homeostatic response to the amount of glucose presently in the blood of an individual, while a high value for A₂/λ represents a strong inertial response of the body based on past levels of blood glucose.

### Female Prediabetic Subphenotypes

The first female prediabetic subphenotype exhibits a cluster center at (0.1901, 0.6970) and relatively low values of A1 and high values of A2/λ. These subjects exhibit a strong inertial response to glucose (past states) and a weak response to present states. This first phenotype reacts more to past states and only moderately to current states of glucose in the blood. Physiologically, these subjects may exhibit some insulin resistance due to over-secretion of insulin and may have fewer insulin receptors but produce insulin normally resulting in a delayed response. Clinical objectives for treatment include improving insulin sensitivity and this prediabetic subphenotype is expected to respond favorably to medications such as metformin.

The second female prediabetic subphenotype exhibits a cluster center at: (0.2769, 0.5075) and relatively low values of A1 and low values of A2/80 . These subjects exhibit a weak instantaneous response to the present state of glucose and a moderate inertial response to past states of glucose in the blood. Physiologically, these subjects may have fewer insulin receptors but may produce insulin normally. This results in a delayed response, but not as delayed as the first female prediabetic subphenotype. Clinical objectives for treatment include improving Insulin sensitivity and insulin production. Subjects with this prediabetic subphenotype should adjust lifestyle factors including adjustments to diet and exercise and monitor blood glucose levels closely to ensure no further disease progression.

The third female prediabetic subphenotype exhibits a cluster center at: (0.4393, 0.4827) and relatively high values of A1 and low values of A2/λ. These subjects exhibit a strong instantaneous response to the present state of glucose and the response is relatively balanced between instantaneous glucose levels and recent history. This control of glucose appears most similar to that observed in healthy patients. Physiologically, these subjects may not exhibit significant insulin resistance and the homeostatic response is fairly “normal”. This prediabetic subphenotype may therefore be associated with prediabetic individuals who are conscious about their diet and exercise regime and limit their glucose intake. Clinical objectives for treatment include continued awareness over diet and exercise and continued monitoring.

### Male Prediabetic Subphenotypes

The first male prediabetic subphenotype exhibits a cluster center at: (0.1175, 0.7216) and relatively low values of A1 and high values of A2/λ. These subjects exhibit a strong inertial response to glucose (past states) and a weak response to present states. This first phenotype reacts more according to past states and only moderately to current states of glucose in the blood. Physiologically, these subjects may exhibit some insulin resistance due to over-secretion of insulin and may have fewer insulin receptors but produce insulin normally resulting in a delayed response. Clinical objectives for treatment include improving insulin sensitivity and this prediabetic subphenotype is expected to respond favorably to medications such as metformin.

The second male prediabetic subphenotype exhibits a cluster center at: (0.1830, 0.5217) and relatively low values of A₁ and low values of A2/λ. These subjects exhibit a weak instantaneous response to the present state of glucose and a moderate inertial response to past states of glucose in the blood. Physiologically, these subjects may have fewer insulin receptors but may produce insulin normally.

This results in a delayed response, but not as delayed as the first male prediabetic subphenotype. Clinical objectives for treatment include improving Insulin sensitivity and insulin production. Subjects with this prediabetic subphenotype should adjust lifestyle factors including adjustments to diet and exercise and monitor blood glucose levels closely to ensure no further disease progression.

The third male prediabetic subphenotype exhibits a cluster center at: (0.3556, 0.4626) and relatively high values of A1 and low values of A2/λ. These subjects exhibit a strong instantaneous response to the present state of glucose and the response is relatively balanced between instantaneous glucose levels and recent history. This control of glucose appears most similar to that observed in healthy patients. Physiologically, these subjects may not exhibit significant insulin resistance and the homeostatic response is fairly “normal”. This prediabetic subphenotype may therefore be associated with prediabetic individuals who are conscious about their diet and exercise regime and limit their glucose intake. Clinical objectives for treatment include continued awareness over diet and exercise and continued monitoring.

The fourth male prediabetic subphenotype exhibits a cluster center at: (0.2886, 0.5787) and relatively high values of A1 and high values of A2/λ. These subjects exhibit a weak instantaneous response to the present state of glucose and a moderate inertial response to past states. These subjects appear to have more homeostatic dysfunction than the third male prediabetic subphenotype but have a very limited occurrence of type 2 diabetic individuals compared to the first and second subtypes. Physiologically, these subject may have fewer insulin receptors but may produce insulin normally. This results in a delayed response, but not as delayed as the response seen in the first female prediabetic subtype. Clinical objectives for treatment include significant lifestyle adjustments to diet and exe rise and blood glucose levels should be monitored closely to ensure no further disease progression.

### Example 5: Gestational Diabetes Study

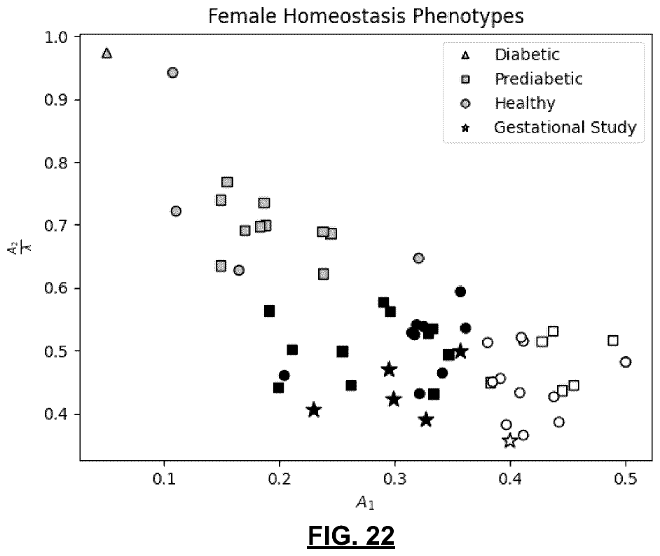

FIG. 22 shows a plot of A₂/λ vs. A₁ for female study subjects. FIG. 22 updates FIG. 19A to show subjects of the gestational diabetes study in view of the existing female subjects in Example 4 (above). Study data for the gestational diabetes study is provided below in Table 9 below.

Blood glucose data was collected for a sample of 6 female subjects using a continuous glucose monitoring (CGM) device (FreeStyle Libre™ CGM) along with demographic and physiological information. Each of the 6 subjects were pregnant at the time of data collection. None of the 6 subjects had gestational diabetes diagnoses.

Representative curves were generated for each subject and then fitted to the model of glycemic control as described in Example 1 resulting in the determination of values for a proportional coefficient A₁, an integral coefficient A₂, an inverse memory time scale λ, a steady depletion coefficient A₃, and a feedback coefficient A₄.

The two primary parameters of the glucose homeostasis model are denoted by A₁, the coefficient of proportionality, and A₂, the integral coefficient or memory coefficient. A₁can be interpreted as the coefficient that controls the instantaneous response of blood glucose fluctuations, and A₂ can be interpreted as a feedback mechanism that accounts for recent fluctuations or alternatively the coefficient that controls the inertial response of blood glucose fluctuations. Therefore it is expected that the values of A₁ and A₂ may decrease as an individual becomes more and more diabetic.

k-means clustering was also used to separate subjects into clusters and prediabetic subphenotypes based on their proximity to cluster centres (as described in Example 4). If the cluster center A₁ and A₂/λ are denoted as A1c and A₂lc, and each individual's parameters points are denoted A1p and A2lp, the distance between the point and the center is calculated as follows:

$$d = \sqrt{{( A 1 c - A 1 p )}^{2} + {( A 2 lc - A 2 lp )}^{2}}$$

The three female prediabetic subphenotypes from Example 4 above may be expressed based on their cluster centers as follow Female Phenotype Center 1 (FP1): (0.43934608, 0.48272659), Female Phenotype Center 2 (FP2): (0.27692836, 0.50758159), Female Phenotype Center 3 (FP3): (0.19006145, 0.69702349).

The “first female prediabetic subphenotype” from Example 4 above exhibiting a cluster center of (0.19006145, 0.69702349) is referred to as Female Phenotype Center 3 (FP3) here in Example 5. The “second female prediabetic subphenotype” from Example 4 above exhibiting a cluster center of (0.27692836, 0.50758159) is referred to as Female Phenotype Center 2 (FP2) for the analysis here in Example 5. The “third female prediabetic subphenotype” from Example 4 above exhibiting a cluster center of (0.43934608, 0.48272659) is referred to as Female Phenotype Center 1 (FP1) for the analysis here in Example 5.

### Results

TABLE 9
Determined values of A₁, A₂/λ, and Closest Phenotype Center
for each of the 6 participants who completed the study.

| | | | Average | | | Closest |
|---|---|---|---|---|---|---|
| | Gestational | CGM | Glucose | | | Phenotype |
| Participant ID | Diabetes? | Length | Level | A1 | A2/λ | Center |
| 0M000K2QMY0 | No | 12 | 4.16 | 0.2993 | 0.4226 | FP2 |
| | | days | mmol/L | | | (0.27692836, |
| | | | | | | 0.50758159) |
| 0M000KD9YDM | No | 14 | 4.52 | 0.2301 | 0.4058 | FP2 |
| | | days | mmol/L | | | (0.27692836, |
| | | | | | | 0.50758159) |
| 0M000KF8100 | No | 14 | 4.85 | 0.3273 | 0.3902 | FP2 |
| | | days | mmol/L | | | (0.27692836, |
| | | | | | | 0.50758159) |
| 0M000KM0WVW | No | 14 | 4.80 | 0.2953 | 0.4699 | FP2 |
| | | days | mmol/L | | | (0.27692836, |
| | | | | | | 0.50758159) |
| 0M000KP1TUR | No | 14 | 4.78 | 0.4000 | 0.3567 | FP1 |
| | | days | mmol/L | | | (0.43934608, |
| | | | | | | 0.48272659) |
| 0M0067KZE08 | No | 14 | 4.74 | 0.3572 | 0.4989 | FP2 |
| | | days | mmol/L | | | (0.27692836, |
| | | | | | | 0.50758159) |

Five of the subjects (shown as stars) in the gestational study were identified as having a closest center of FP2. One of the subjects (shown as a shaded star) in the gestational study was identified as having a closest center of FP1.

The distribution of phenotypes in the results of Example 5, while reflecting a small sample, shows that a predisposition to gestational diabetes is correlated with the Female Phenotype Center 2 (FP2).

All references cited herein are hereby incorporated by reference in their entirety.

### REFERENCES

Ahlqvist, E. et al. Novel subgroups of adult-onset diabetes and their association with outcomes: a data-driven cluster analysis of six variables. Lancet Diabetes. Endocrinol. 6, 361-369 (2018).
American Diabetes Association (2018). Statistics About Diabetes. Available from https://www.diabetes.org/resources/statistics/statistics-about-diabetes
Bergman RN, Ider YZ, Bowden CR, Cobelli C (1979) Quantitative estimation of insulin sensitivity. Am J Physiol 236: E667-E677.
Bergman, R. N., and C. Cobelli. (1980). Minimal modeling, partition analysis, and the estimation of insulin sensitivity. Fed. Proc. 39: 110-115.
Brüssow, H. (2013). What is health? Microbial Biotechnology 6: 341-348.
Centers for Disease Control and Prevention (2017). National Diabetes Statistics Report, 2017. Available from https://www.cdc.gov/diabetes/pdfs/data/statistics/national-diabetes-statistics-report.pdf
Dennis, J. M., Shields, B. M., Henley, W. E., Jones, A. G. & Hattersley, A. T. Disease progression and treatment response in data-driven subgroups of type 2 diabetes compared with models based on simple clinical features: an analysis using clinical trial data. Lancet Diabetes Endocrinol. 7, 442-451 (2019).
Handelsman Y., et al. (2015) American Association of Clinical Endocrinologists and American College of Endocrinology: clinical practice guidelines for developing a diabetes mellitus comprehensive care plan. Endocr Pract. 21:1-87.
Kotas, M. E. & Medzhitov, R. (2015). Homeostasis, inflammation, and disease susceptibility. Cell 160, 816-827
Masroor, S. et al. (2019) Mathematical modeling of the glucagon challenge test J. Pharmacokinet. Phar. https://doi.org/10.1007/s10928-019-09655-2
Stidsen, J. V. et al. Pathophysiology-based phenotyping in type 2 diabetes: a clinical classification tool. Diabetes Metab. Res. Rev. 0, e3005 (2018).
Wagner, R., Heni, M., Tabák, A.G. et al. Pathophysiology-based subphenotyping of individuals at elevated risk for type 2 diabetes. Nat Med 27, 49-57 (2021). https://doi.org/10.1038/841591-020-1116-9

1. A method for classifying a subject into a glucose homeostasis phenotype, the method comprising:
generating a glucose homeostasis model for the subject based on a plurality of glucose measurements for the subject; wherein the glucose homeostasis model comprises a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate;
classifying the subject into the glucose homeostasis phenotype based on one or more of A₁, A₂, A, A₃ and A₄.

2. The method of claim 1, comprising classifying the subject based on a value for A₁.

3. The method of claim 2, wherein the subject is a male subject and a subject with a value of A₁ less than 0.25 is classified as having a weak instantaneous glucose response phenotype or wherein the subject is a female subject and a subject with a value A₁ less than 0.30 is classified as having a poor instantaneous glucose response phenotype.

4. The method of claim 3, comprising classifying the subject based on a value for A₂/λ.

5. The method of claim 4, wherein a value of A₂/λ less than 0.6 is indicative of a weak inertial glucose response phenotype.

6. The method of claim 5, wherein the subject is pre-diabetic.

7. The method of claim 6, wherein the subject has one or more of a HbA1c between 5.7 and 6.4, an Oral Glucose Tolerance Test (OGTT) between 140 mmol/ L and 200 mmol/ L and a Resting Blood Glucose (RBG) between 100 mmol/L and 125 mmol/L.

8. The method of claim 7, wherein:
a. a female subject with a value of A₁ less than 0.3 and a value of A₂/λ greater than 0.6 is classified as having a first prediabetic subphenotype;
b. a female subject with a value of A₁ less than 0.3 and a value of A₂/λ less than 0.6 is classified as having a second prediabetic subphenotype; and
C. a female subject with a value of A₁ greater than 0.3 and a value of A₂/λ less than 0.6 is classified as having a third prediabetic subphenotype.

9. The method of claim 8, further comprising selecting the female subject with the first prediabetic subphenotype for treatment to improve insulin sensitivity, optionally for treatment with metformin.

10. The method of claim 7, wherein:
a. a male subject with a value of A₁ less than 0.25 and a value of A₂/λ greater than 0.6 is classified as having a first prediabetic subphenotype;
b. a male subject with a value of A₁ less than 0.25 and a value of A₂/λ less than 0.6 is classified as having a second prediabetic subphenotype;
C. a male subject with a value of A₁ greater than 0.3 and a value of A₂/λ less than 0.6 is classified as having a third prediabetic subphenotype; and
d. a male subject with a value of A₁ greater than 0.3 and a value of A₂/λ greater than 0.6 is classified as having a fourth prediabetic subphenotype.

11. The method of claim 10, further comprising selecting the male subject with the first prediabetic subphenotype for treatment to improve insulin sensitivity, optionally for treatment with metformin.

12. The method of claim 11, further comprising treating or monitoring the subject based on the classification of the subject into the glucose homeostasis phenotype, optionally based on the prediabetic subphenotype.

13. The method of claim 12, wherein generating the glucose homeostasis model for the subject comprises:
receiving, at a processor, a plurality of glucose measurements for the patient, the plurality of glucose measurements for the patient comprising a time-series collected from the patient using a glucose measurement device;
selecting, at the processor, one or more curve intervals in the plurality of glucose measurements, the one or more curve intervals corresponding to one or more local maxima of the plurality of glucose measurements;
determining, at the processor, a representative curve based on the one or more curve intervals;
determining, at the processor, a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate;
generating, at the processor, the glucose homeostasis model, the glucose homeostasis model comprising the proportional coefficient A₁, the integral coefficient A₂, the inverse memory time scale λ, the steady depletion coefficient A₃, and the feedback coefficient A₄.

14. The method of claim 13 further comprising determining, at the processor, a glucose homeostasis metric based on one or more of the group of the proportional coefficient A₁, the integral coefficient A₂, the steady depletion coefficient A₃, the feedback coefficient A₄, and the inverse memory time scale term λ, wherein the glucose homeostasis metric is A₁ or A₂/λ.

15.-19. (canceled)

20. A system for classifying a subject into a glucose homeostasis phenotype, the system comprising:
a memory, the memory comprising a plurality of glucose measurements for the patient, the plurality of glucose measurements for the patient comprising a time-series collected from the patient using a glucose measurement device;
a processor in communication with the memory, the processor configured to:
select one or more curve intervals in the plurality of glucose measurements, the one or more curve intervals corresponding to one or more local maxima of the plurality of glucose measurements;
determine a representative curve based on the one or more curve intervals;
determine a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate;
generate the glucose homeostasis model, the glucose homeostasis model comprising the proportional coefficient A₁, the integral coefficient A₂, the inverse memory time scale 80 , the steady depletion coefficient A₃, and the feedback coefficient A₄.
classify the subject into the glucose homeostasis phenotype based on one or more of based on one or more of A₁, A₂, λ, A₃ and A₄.

21. (canceled)

22. The system of claim 20, wherein the processor is configured to generate the glucose homeostasis model for the subject by:
receiving a plurality of glucose measurements for the patient, the plurality of glucose measurements for the patient comprising a time-series collected from the patient using a glucose measurement device;
selecting one or more curve intervals in the plurality of glucose measurements, the one or more curve intervals corresponding to one or more local maxima of the plurality of glucose measurements;
determining a representative curve based on the one or more curve intervals;
determining a proportional coefficient A₁ for response of a controller u(t) to an error e(t), an integral coefficient A₂ for response of the controller u(t) to past values of error e(t), an inverse memory time scale λ for decay of an integral term, a steady depletion coefficient A₃ for a basic metabolic rate, and a feedback coefficient A₄ for an approximate mass action rate;
generating the glucose homeostasis model, the glucose homeostasis model comprising the proportional coefficient A₁, the integral coefficient A₂, the inverse memory time scale λ, the steady depletion coefficient A₃, and the feedback coefficient A₄.

23. The system of claim 22 wherein the processor is further configured to determine a glucose homeostasis metric based on one or more of the group of the proportional coefficient A₁, the integral coefficient A₂, the steady depletion coefficient A₃, the feedback coefficient A₄, and the inverse memory time scale term λ, wherein the glucose homeostasis metric is A₁ or A₂/λ.

24. The system of claim 23 wherein the processor is further configured to determine a glucose homeostasis metric based on one or more of the group of the proportional coefficient A₁, the integral coefficient A₂, the steady depletion coefficient A₃, the feedback coefficient A₄, and the inverse memory time scale term λ, wherein the glucose homeostasis metric is A₁ or A₂/λ.

25. The system of claim 24, wherein the plurality of glucose measurements are received from a glucose measurement device and optionally wherein the glucose measurement device is a FreeStyle™ Libre.
